(19) United States
(12) Patent Application Publication
(10) Pub. No.: US 2020/0190202 A1
(43) Pub. Date: Jun. 18, 2020

(54) Reprogramming of CD8 T Cells with CXCL12 Signaling Inhibitors

(71) Applicant: The General Hospital Corporation, Boston, MA (US)

(72) Inventor: Mark C. Poznansky, Newton Center, MA (US)

(21) Appl. No.: 16/637,972

(22) PCT Filed: Aug. 10, 2018
(86) PCT No.: PCT/US2018/046202

§ 371 Date: Feb. 10, 2020

Related U.S. Application Data
(60) Provisional application No. 62/544,339, filed on Aug. 11, 2017.
(60) Provisional application No. 62/609,605, filed on Dec. 22, 2017.

(51) Int. Cl. C07K 16/28; A61K 31/395; A61P 35/00

(52) U.S. Cl. CPC C07K 16/2866; A61K 31/395; A61K 45/06; C07K 16/2818; A61P 35/00; C07K 16/2809

(57) ABSTRACT

The present invention relates to methods and compositions for decreasing the level of PD-1 on a CD8⁺ T cell, converting a CD25⁺ Foxp3⁺ regulatory T cell to a CD25⁻ Foxp3⁻ helper-like T cell, and reprogramming subpopulations of T cells to a phenotype suitable to enhance an immunotherapy treatment using an inhibitor of CXCL12 signaling.

### STATEMENT OF PRIORITY

This application claims the benefit of U.S. Provisional Application Ser. No. 62/609,605, filed Dec. 22, 2017, and U.S. Provisional Application Ser. No. 62/544,339, filed Aug. 11, 2017, the entire contents of each of which are incorporated by reference herein.

### FIELD OF THE INVENTION

The present invention relates to methods and compositions for decreasing the level of PD-1 on a CD8⁺ T cell, converting a CD25⁺ Foxp3⁺ regulatory T cell to a CD25⁻ Foxp3⁺ helper-like T cell, and reprogramming subpopulations of T cells to a phenotype suitable to enhance an immunotherapy treatment using an inhibitor of CXCL12 signaling (e.g., CXCR4 and/or CXCR7 antagonist).

### BACKGROUND OF THE INVENTION

Malignant mesothelioma (MM) is an aggressive tumor that arises from the pleural and peritoneal mesothelium. MM is largely refractory to conventional therapies and the median survival after symptom onset is often less than 12 months (1-4). Surgery, radiotherapy and chemotherapy have improved quality of life but have made little impact on survival with this tumor. Studies over the last two decades suggest that immunotherapy may be a promising avenue for the treatment of MM given that the tumor expresses antigens that can be targeted by the immune system (5-7). Although clinical trials of various immunotherapeutic modalities have not yet yielded significant benefit (8), the failures to date can be understood in light of the complexities of intratumoral immune dysregulation, which require the development of more efficacious combination immunotherapies that address these immune evasion mechanisms.

A novel fusion protein was previously described (9), consisting of the broadly immune activating Mycobacterium tuberculosis-derived heat shock protein 70 (MtbHsp70) and the tumor antigen targeting activity of a single-chain variable fragment (scFv) binding mesothelin (MSLN), a validated immunotherapy target (10-12). The antitumor efficacy of this MSLN-targeted fusion protein as an in vivo vaccination strategy was evaluated in syngeneic immunocompetent mouse models of ovarian cancer and mesothelioma and demonstrated that this bifunctional fusion protein significantly enhances survival and slows tumor growth through the augmentation of tumor-specific cell-mediated immune responses (9). The current version of this fusion protein, VIC-008, was derived from the original by slight modifications that remove redundant amino acids, and introduce a single amino acid mutation, phenylalanine to valine, at position 381 of MtbHsp70 to prevent non-specific peptide binding and presentation while retaining immune-stimulatory capacity. This fusion protein showed significantly improved efficacy in tumor control and animal survival in a mouse model of MSLN-expressing ovarian cancer over the original protein (13). However, the intratumoral immunosuppressive microenvironment, including most notably the presence of regulatory T cells (T_{reg}), may limit the effectiveness of VIC-008. Removal of T_{reg} cells has been shown to result in tumor growth inhibition and the release of antitumor effector T cells from immunosuppression (14).

The critical role of the chemokine receptor 4 (CXCR4) and its ligand (CXCL12) in the pathogenesis of many tumors has been well recognized (15, 16). AMD3100, a specific antagonist for CXCR4, was originally developed as an anti-HIV drug (17), and later applied as a reagent to mobilize hematopoietic stem cells from bone marrow (18). A number of studies have shown that AMD3100 can impact tumor growth, metastasis and angiogenesis by blockade of the CXCL12/CXCR4 axis (19-22). It has previously been reported that the blockade of CXCL12/CXCR4 axis with AMD3100 as a monotherapy in ovarian tumor-bearing mice conferred a survival advantage and elicited multimodal effects on tumor pathogenesis, including selective reduction of intratumoral T_{reg} cells (23). However, the precise mechanism of AMD3100-mediated immunomodulation remains unknown.

Immunomodulators have been widely used in combination with tumor vaccines or immunotherapies for improving antitumor immune responses, which include removing or inhibiting suppressive cells such as T_{reg} cells, regulatory type II NKT cells, or myeloid-derived suppressor cells (MDSC) (24-29).

However, there is a need in the art for improved immunotherapy treatments for diseases that can overcome the immunosuppressive barrier provided by many tumors and disease cells and enhance the ability of the immune system to recognize and attack the cells of such diseases.

### SUMMARY OF THE INVENTION

The present invention is based, in part, on the finding that treatment with a CXCL12 signaling inhibitor enhanced the effects of VIC-008 in tumor control and animal survival in two syngeneic orthotopic models of MM in immunocompetent mice, and this response was associated with CXCL12 signaling inhibitor-mediated neutralization of intratumoral immunosuppression. In addition, the present invention is based, in part, on the determination of a novel mechanism by which inhibitors of CXCL12 signaling modulate immunity alone and in combination with VIC-008.

Accordingly, one aspect of the invention relates to a method of decreasing the level of PD-1 on a CD8⁺ T cell, comprising contacting the T cell with an amount of an inhibitor of CXCL12 signaling (e.g., a CXCR4 antagonist and/or CXCR7 antagonist) effective to decrease the level of PD-1 on the CD8⁺ T cell.

Another aspect of the invention relates to a method of converting a CD25⁺ Foxp3⁺ regulatory T cell to a CD25⁻ Foxp3⁺ helper-like T cell, comprising contacting the T cell with an amount of an inhibitor of CXCL12 signaling (e.g., a CXCR4 antagonist and/or CXCR7 antagonist) effective to convert the CD25⁺ Foxp3⁺ regulatory T cell to a CD25⁻ Foxp3⁺ helper-like T cell.

A further aspect of the invention relates to a method of reprogramming subpopulations of T cells to a phenotype suitable to enhance an immunotherapy treatment in a subject, comprising contacting T cell subpopulations with an amount of an inhibitor of CXCL12 signaling (e.g., a CXCR4 antagonist and/or CXCR7 antagonist) effective to reprogram the subpopulations of T cells to a phenotype suitable to enhance an immunotherapy treatment.

An additional aspect of the invention relates to a composition comprising an inhibitor of CXCL12 signaling (e.g., a CXCR4 antagonist and/or CXCR7 antagonist) for use in decreasing the level of PD-1 on a CD8⁺ T cell, comprising contacting the T cell with an amount of a CXCR4 and/or CXCR7 antagonist effective to decrease the level of PD-1 on the CD8⁺ T cell.

Another aspect of the invention relates to a composition comprising an inhibitor of CXCL12 signaling (e.g., a CXCR4 antagonist and/or CXCR7 antagonist) for use in converting a CD25⁺ Foxp3⁺ regulatory T cell to a CD25⁻ Foxp3⁺ helper-like T cell.

A further aspect of the invention relates to a composition comprising an inhibitor of CXCL12 signaling (e.g., a CXCR4 antagonist and/or CXCR7 antagonist) for use in reprogramming subpopulations of T cells to a phenotype suitable to enhance an immunotherapy treatment in a subject.

An additional aspect of the invention relates to the use of an inhibitor of CXCL12 signaling (e.g., a CXCR4 antagonist and/or CXCR7 antagonist) in the preparation of a medicament for decreasing the level of PD-1 on a CD8⁺ T cell, comprising contacting the T cell with an amount of an inhibitor of CXCL12 signaling (e.g., a CXCR4 antagonist and/or CXCR7 antagonist) effective to decrease the level of PD-1 on the CD8⁺ T cell.

Another aspect of the invention relates to the use of an inhibitor of CXCL12 signaling (e.g., a CXCR4 antagonist and/or CXCR7 antagonist) in the preparation of a medicament for converting a CD25⁺ Foxp3⁺ regulatory T cell to a CD25⁻ Foxp3⁺ helper-like T cell.

A further aspect of the invention relates to the use of an inhibitor of CXCL12 signaling (e.g., a CXCR4 antagonist and/or CXCR7 antagonist) in the preparation of a medicament for reprogramming subpopulations of T cells to a phenotype suitable to enhance an immunotherapy treatment in a subject.

These and other aspects of the invention are set forth in more detail in the description of the invention below.

### BRIEF DESCRIPTION OF THE DRAWINGS

FIGS. 1A-1D show tumor growth and mice survival in different treatment groups. All of the intraperitoneal tumors were collected at day 7 after the last treatment and weighed in 40 L (n=10) (A) and AE17 (n=10) (B) mice. The survival of 40 L (n=12) (C) and AE17 (n=12) (D) tumor bearing mice. Numbers on the X axis represented days of mouse survival after inoculation of tumor cells into mice. NS, not significant, *P<0.05, ** P<0.01, ***P<0.001 and ****P<0.0001. Data were presented as mean±SEM.

FIGS. 2A-2F show VIC-008 facilitates lymphocyte infiltration. (A) Gating strategy for infiltrated lymphocytes. The proportion of CD8⁺ T cells in total live splenocytes in 40 L (n=6) (B) and AE17 (n=5) (C) mice. The proportion of CD8⁺ T cells in total live cells in lymph nodes of AE17 mice (n=5) (D). The proportion of CD8⁺ T cells in total live cells in tumors of 40 L (n=6) (E) and AE17 (n=5) (F) mice. *P<0.05, **P<0.01 and ***P<0.001. Data are presented as mean±SEM.

FIGS. 3A-3D show VIC-008 promoted CD8⁺ T-cell IFN-γ secretion. (A) Representative dot plots of IFN-γ secreting cells in different treatment groups. The proportion of IFN-γ secreting cells in CD8⁺ T cells in spleens of 40 L (n=6) (B) and AE17 (n=5) (C) mice. The proportion of IFN-γ secreting cells in CD8⁺. T cells in lymph nodes of AE17 mice (n=5) (D). **P<0.01 and ***P<0.001. Data are presented as mean±SEM.

FIGS. 4A-4E show AMD3100 decreased PD-1 expression on CD8⁺ T cells. The proportion of PD-1-expressing cells in CD8⁺ T cells in spleens of 40 L (n=6) (A) and AE17 (n=5) (B) mice. The proportion of PD-1-expressing cells in CD8⁺ T cells in lymph nodes of AE17 mice (n=5) (C). The proportion of PD-1-expressing cells in CD8⁺ T cells in tumors of 40 L (n=6) (D) and AE17 (n=5) (E) mice. *P<0.05 and **P<0.01. Data are presented as mean±SEM.

FIGS. 5A-5F show AMD3100 reduced tumor-infiltrating T_{reg}. The proportion of T_{reg} in total live splenocytes in 40 L (n=6) (A) and AE17 (n=5) (B) mice. The proportion of T_{reg} in total live cells in lymph nodes of AE17 mice (n=5) (C). The ratio of CD8⁺ T cells to T_{reg} in lymph nodes of AE17 mice (n=5) (D). The proportion of T_{reg} in total live cells in tumors of 40 L mice (n=6) (E). The ratio of CD8⁺ T cells to T_{reg} in tumors of 40 L mice (n=6) (F). *P<0.05, **P<0.01, ***P<0.001 and ****P<0.0001. Data are presented as mean±SEM.

FIGS. 6A-6E show AMD3100 reprogrammed T_{reg} to helper-like cells. The ratio of CD25⁻ to CD25⁺ cells in CD4⁺ Foxp3⁺ T-cell population in tumors of 40 L (n=6) (A) and in lymph nodes of AE17 (n=5) (B) mice. Representative density plots of IL-2⁺ CD40L⁺ cells in Foxp3⁺ CD25⁻ T_{reg} population in different treatment groups (C). The proportion of IL-2⁺ CD40L⁺ cells in Foxp3⁺ CD25⁻ T_{reg} population in tumors of 40 L (n=6) (D) and in lymph nodes of AE17 (n=5) (E) mice. *P<0.05, **P<0.01, ***P<0.001 and ****P<0.0001. Data are presented as mean±SEM.

FIGS. 7A-7F show AMD3100-driven T_{reg} reprogramming required TCR activation. Representative dot plots of Foxp3⁺ CD25⁻ and Foxp3⁺ CD25⁺ population with or without AMD3100 treatment under no anti-CD3/CD28 stimulation (A) and statistical difference was analyzed using unpaired t-test with Welch's correction (n=4) (B). Representative dot plots of Foxp3⁺ CD25⁻ and Foxp3⁺ CD25⁺ population with or without AMD3100 treatment under anti-CD3/CD28 stimulation (C) and statistical difference was analyzed using unpaired t-test with Welch's correction (n=4) (D). Representative density plots of IL-2⁺ CD40L⁺ cells in Foxp3⁺ CD25⁻ T_{reg} population with or without AMD3100 treatment under anti-CD3/CD28 stimulation (E) and statistical difference was analyzed using unpaired t-test with Welch's correction (n=4) (F). Data are presented as mean±SEM.

### DETAILED DESCRIPTION OF THE INVENTION

The present invention will now be described in more detail with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of skill in the art to which this invention belongs. The terminology used in the description of the invention herein is for the purpose of describing particular embodiments only and is not intended to be limiting of the invention. All publications, patent applications, patents, patent publications and other references cited herein are incorporated by reference in their entireties for the teachings relevant to the sentence and/or paragraph in which the reference is presented.

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

To illustrate, if the specification states that a complex comprises components A, B and C, it is specifically intended that any of A, B or C, or a combination thereof, can be omitted and disclaimed singularly or in any combination.

As used in the description of the invention and the appended claims, the singular forms “a,” “an,” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of polypeptide, dose, time, temperature, enzymatic activity or other biological activity and the like, is meant to encompass variations of ±10%, ±5%, ±1%, ±0.5%, or even ±0.1% of the specified amount.

As used herein, the transitional phrase “consisting essentially of” (and grammatical variants) is to be interpreted as encompassing the recited materials or steps and those that do not materially affect the basic and novel characteristic(s) of the claimed invention. Thus, the term “consisting essentially of as used herein should not be interpreted as equivalent to “comprising.”

The term “modulate,” “modulates,” or “modulation” refers to enhancement (e.g., an increase) or inhibition (e.g., a decrease) in the specified level or activity.

The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, or even fifteen-fold.

The term “inhibit” or “reduce” or grammatical variations thereof as used herein refers to a decrease or diminishment in the specified level or activity of at least about 15%, 25%, 35%, 40%, 50%, 60%, 75%, 80%, 90%, 95% or more. In particular embodiments, the inhibition or reduction results in little or essentially no detectible activity (at most, an insignificant amount, e.g., less than about 10% or even 5%).

The term “contact” or grammatical variations thereof as used with respect to a cell and an amount of an inhibitor of CXCL12 signaling (e.g., a CXCR4 antagonist and/or CXCR7 antagonist) refers to bringing the inhibitor (e.g., antagonist) and the cell in sufficiently close proximity to each other for the antagonist to exert a biological effect on the cell. In some embodiments, the term “contact” means binding of the inhibitor to the cell. In some embodiments, the term “contact” can mean co-incubating the cell and an inhibitor of CXCL12 signaling (e.g., a CXCR4 antagonist and/or CXCR7 antagonist).

A “therapeutically effective” amount as used herein is an amount that provides some improvement or benefit to the subject. Alternatively stated, a “therapeutically effective” amount is an amount that will provide some alleviation, mitigation, or decrease in at least one clinical symptom in the subject. Those skilled in the art will appreciate that the therapeutic effects need not be complete or curative, as long as some benefit is provided to the subject.

By the terms “treat,” “treating,” or “treatment of,” it is intended that the severity of the subject's condition is reduced or at least partially improved or modified and that some alleviation, mitigation or decrease in at least one clinical symptom is achieved.

“Antibodies” as used herein include polyclonal, monoclonal, single chain, chimeric, humanized and human antibodies, prepared according to conventional methodology.

The terms “vaccine,” “vaccination,” and “immunization” are well-understood in the art, and are used interchangeably herein. For example, the terms vaccine, vaccination, or immunization can be understood to be a process or composition that increases a subject's immune reaction to an immunogen (e.g., by providing an active immune response), and therefore its ability to resist, overcome and/or recover from infection (i.e., a protective immune response) and/or target cancer cells or other disease cells.

A first aspect of the invention relates to a method of decreasing the level of PD-1 on a CD8⁺ T cell, comprising contacting the T cell with an amount of an inhibitor of CXCL12 signaling (e.g., a CXCR4 antagonist and/or CXCR7 antagonist) effective to decrease the level of PD-1 on the CD8⁺ T cell. In some embodiments, the level of PD-1 on a CD8⁺ T cell is decreased as compared to a T cell that has not been contacted with the inhibitor of CXCL12 signaling. In some embodiments, a CD8⁺ T cell may be an in vitro or ex vivo cell. In some embodiments, the CD8⁺ T cell may be in a subject. In some embodiments, the inhibitor may downregulate the expression of PD-1. In some embodiments, contacting a T cell with an amount of an inhibitor of CXCL12 signaling (e.g., a CXCR4 antagonist and/or CXCR7 antagonist) may comprise co-incubating the T cell and the inhibitor. In further embodiments, contacting a T cell with an amount of an inhibitor of CXCL12 signaling (e.g., a CXCR4 antagonist and/or CXCR7 antagonist) may comprise administering the inhibitor to a subject. In some embodiments, an inhibitor may be administered to a subject systemically and/or locally.

A further aspect of the invention provides a method of converting a CD25⁺ Foxp3⁺ regulatory T cell to a CD25⁻ Foxp3⁺ helper-like T cell, comprising contacting the CD25⁺ Foxp3⁺ regulatory T cell with an amount of an inhibitor of CXCL12 signaling (e.g., a CXCR4 antagonist and/or CXCR7 antagonist) effective to convert the CD25⁺ Foxp3⁺ regulatory T cell to a CD25⁻ Foxp3⁺ helper-like T cell. In some embodiments, a helper-like T cell may be CD25⁻ Foxp3⁺ IL-2⁺ CD40L⁺. In some embodiments, the method may further comprise activating a T cell receptor on the regulatory T cell. In some embodiments, activating a T cell receptor may comprise contacting the regulatory T cell with an anti-CD3/CD28 antibody. In some embodiments, contacting the CD25⁺ Foxp3⁺ regulatory T cell comprises co-incubating the regulatory T cell and the inhibitor of CXCL12 signaling In some embodiments, a regulatory T cell may be an in vitro or ex vivo cell. In further embodiments, the regulatory T cell may be in a subject. In some embodiments, contacting the regulatory T cell with an amount of an inhibitor of CXCL12 signaling (e.g., a CXCR4 antagonist and/or CXCR7 antagonist) may comprise administering the inhibitor to a subject. In some embodiments, an inhibitor of CXCL12 signaling may be administered to a subject systemically and/or locally.

An additional aspect of the invention relates to a method of reprogramming subpopulations of T cells to a phenotype suitable to enhance an immunotherapy treatment in a subject, comprising contacting T cell subpopulations with an amount of inhibitor of CXCL12 signaling (e.g., a CXCR4 antagonist and/or CXCR7 antagonist) effective to reprogram the subpopulations of T cells to a phenotype suitable to enhance an immunotherapy treatment. As used herein, “suitable to enhance an immunotherapy treatment” means that the reprogrammed subpopulations of T cells when administered (or reprogrammed in vivo) to a subject receiving or having received immunotherapy treatment, impart an improvement over the immunotherapy treatment such that the severity of the subject's condition is reduced or at least partially improved or modified and that some alleviation, mitigation or decrease in at least one clinical symptom is achieved as compared to the condition of a subject receiving or having received immunotherapy treatment but which has not been administered the reprogrammed subpopulations of T cells or for which the T cells of the subject have not been reprogrammed in vivo. In some embodiments, a T cell subpopulation may be in vitro or ex vivo. In some embodiments, T cell subpopulations may be obtained from a subject, reprogrammed, and administered to the subject. In some embodiments, contacting T cell subpopulations may comprise co-incubating the T cell subpopulations and an inhibitor of CXCL12 signaling (e.g., a CXCR4 and/or CXCR7 antagonist). In additional embodiments, the T cell subpopulations may be in a subject. Thus, in some embodiments, contacting the T cell subpopulations may comprise administering an inhibitor of CXCL12 signaling (to the subject. In some embodiments, the inhibitor of CXCL12 signaling may be administered systemically and/or may be administered locally.

In some embodiments, to decrease the level of PD-1 on the CD8⁺ T cell, to convert a CD25⁺ Foxp3⁺ regulatory T cell to a CD25⁻ Foxp3⁺ helper-like T cell, or to reprogram subpopulations of T cells to a phenotype suitable to enhance an immunotherapy treatment, a CXCL12 signaling inhibitor may be administered to a subject that has undergone immunotherapy treatment and the treatment has failed. In some embodiments, the CXCL12 signaling inhibitor may be administered to a subject that is already undergoing immunotherapy treatment. In some aspects, the CXCL12 signaling inhibitor may be administered to a subject at about the same time as the immunotherapy treatment is initiated (e.g., concurrently or about 5 min to about 60 min or more of each other, e.g., about 5, 10, 15, 20, 25, 30, 35, 40, 45, 50, 55, 60 min or more of each other). In some aspects, the CXCL12 signaling inhibitor may be administered to a subject prior to the initiation of immunotherapy treatment (e.g., about 1 day to 1 week or more prior to initiation of immunotherapy treatment, e.g., about 1, 2, 3, 4, 5, 6, 7 days or more).

In some embodiments, a subject that comprises a CD8⁺ T cell for which the level of PD-1 may be decreased, a CD25⁺ Foxp3⁺ regulatory T cell that may be converted to a CD25⁻ Foxp3⁺ helper-like T cell, and/or a subpopulation of T cells that may be reprogrammed to a phenotype suitable to enhance an immunotherapy treatment, may be a subject in need of or undergoing immunotherapy, or may be a subject that has already undergone immunotherapy treatment, which has failed.

In some embodiments, a subject may have cancer. Non-limiting examples of the types of cancer a subject may have include malignant mesothelioma, melanoma (e.g., metastatic melanoma), germ cell cancer, head and neck cancer (e.g., squamous cell carcinoma), oral cancer, lung cancer (e.g., non-small cell lung cancer, e.g., squamous non-small cell lung cancer), bladder cancer, rectal cancer, anal cancer, ovarian cancer, uterine endometrial cancer, uterine sarcoma, brain cancer (e.g., Glioblastoma), esophageal cancer, pancreatic cancer, gastric cancer, hepatocellular carcinoma, renal carcinoma, urothelial cancer, breast cancer, Merkel cell carcinoma, thyroid cancer, non-Hodgkin's lymphoma, chronic lymphocytic leukemia, Hodgkin's lymphoma, multiple myeloma, folicular lymphoma, diffuce large B-cell pymphoma, peripheral T-cell lymphoma, mycosis fungoides, and acute myeloid leukemia.

In some embodiments, the subject may have an infectious disease. In some embodiments, the infectious disease may be a chronic infectious disease or an acute infectious disease. In some embodiments, the infectious disease may be a viral, bacterial, or parasitic infection. A parasitic infection can include an infection by Schistosoma spp. (e.g., S. mansoni), Fasciola spp. (e.g., F. hepatica), Heligmosomoides spp. (e.g., H. polygyrus), Leishmania spp. (e.g., L. mexicana), Toxoplasma spp. (e.g., T. gondii) and Plasmodium spp. (e.g., P. falciparum). Non-limiting examples of an infectious disease can include human immunodeficiency virus (HIV), tuberculosis, malaria, hepatitis (e.g., hepatitis B, hepatitis C), cytomegalovirus (CMV) viraemia.

In some embodiments, a method of decreasing the level of PD-1 on a CD8⁺ T cell, a method of converting a CD25⁺ Foxp3⁺ regulatory T cell to a CD25⁻ Foxp3⁺ helper-like T cell, and/or a method of reprogramming subpopulations of T cells to a phenotype suitable to enhance an immunotherapy treatment may further comprise administering to the subject an immunotherapy or vaccine treatment (e.g., an immune checkpoint inhibitor, therapeutic antibody, etc).

In some embodiments, the invention further provides a composition comprising an inhibitor of CXCL12 signaling (e.g., a CXCR4 and/or CXCR7 antagonist) for use in decreasing the level of PD-1 on a CD8⁺ T cell, comprising contacting the T cell with an amount of an inhibitor of CXCL12 signaling effective to decrease the level of PD-1 on the CD8⁺ T cell.

In some embodiments, the invention provides a composition an inhibitor of CXCL12 signaling (e.g., a CXCR4 and/or CXCR7 antagonist) for use in converting a CD25⁺ Foxp3⁺ regulatory T cell to a CD25⁻ Foxp3⁺ helper-like T cell.

In some embodiments, the invention provides a composition comprising an inhibitor of CXCL12 signaling (e.g., a CXCR4 and/or CXCR7 antagonist) for use in reprogramming subpopulations of T cells to a phenotype suitable to enhance an immunotherapy treatment in a subject.

In some embodiments, the use of an inhibitor of CXCL12 signaling (e.g., a CXCR4 and/or CXCR7 antagonist) in the preparation of a medicament for decreasing the level of PD-1 on a CD8⁺ T cell is provided, comprising contacting the T cell with an amount of an inhibitor of CXCL12 signaling effective to decrease the level of PD-1 on the CD8⁺ T cell.

In some embodiments, the use of an inhibitor of CXCL12 signaling (e.g., a CXCR4 and/or CXCR7 antagonist) in the preparation of a medicament is provided for converting a CD25⁺ Foxp3⁺ regulatory T cell to a CD25⁻ Foxp3⁺ helper-like T cell.

In some embodiments, the use of an inhibitor of CXCL12 signaling (e.g., a CXCR4 and/or CXCR7 antagonist) in the preparation of a medicament is provided for reprogramming subpopulations of T cells to a phenotype suitable to enhance an immunotherapy treatment in a subject.

“CXCL12 signaling” as used herein refers to the signaling pathways that involve binding of CXCL12 to its receptors, including CXCR4 and CXCR7.

An inhibitor of CXCL12 signaling may be any molecule that inhibits the CXCL12/CXCR4/CXCR7 axis. The inhibitor may completely or partially inhibit signaling through the CXCL12/CXCR4/CXCR7 axis when administered to a subject, e.g., providing at least about 30%, 40%, 50%, 60%, 70%, 80%, 90% or more inhibition. Inhibitors may include, without limitation, molecules that inhibit expression of CXCL12 or CXCR4 or CXCR7 (e.g., antisense or siRNA molecules), molecules that bind to CXCL12 or CXCR4 or CXCR7 and inhibit their function (e.g., antibodies or aptamers), molecules that inhibit dimerization of CXCL12 or CXCR4 or CXCR7, and antagonists of CXCR4 or CXCR7. In one embodiment, the inhibitor of CXCL12 signaling is a CXCR4 antagonist.

“CXCR4 antagonist” or “CXCR7 antagonist” refers to a compound that antagonizes CXCL12 binding to CXCR4 and/or CXCR7 or otherwise reduces the chemorepellant effect of CXCL12. Thus, in some embodiments, a CXCR4 antagonist and/or a CXCR7 antagonist may be any molecule that blocks the CXCR4 receptor or the CXCR7 receptor, respectively. Blocking the CXCR4 receptor and/or the CXCR7 receptor prevents CXCL12 from binding to the same. A CXCR4 antagonist and/or CXCR7 antagonist may provide complete or partial inhibition of signaling through the CXCL12/CXCR4/CXCR7 axis (i.e., an inhibitor of CXCL12 signaling) when contacted with a T cell (e.g., a CD8⁺ T cell, a CD25⁺ Foxp3⁺ regulatory T cell, and the like) and/or administered to a subject comprising the T cells, e.g., providing at least about 30%, 40%, 50%, 60%, 70%, 80%, 90% or more inhibition.

In some embodiments, a CXCR4 antagonist can include, but is not limited to, an agent that inhibits binding of CXCL12 to CXCR4. In some embodiments, a CXCR4 antagonist can include, but is not limited to, AMD3100, AMD11070 (also called AMD070), AMD12118, AMD11814, AMD13073, FAMD3465, C131, BKT140, CTCE-9908, KRH-2731, TC14012, KRH-3955, BMS-936564/MDX-1338, LY2510924, GSK812397, KRH-1636, T-20, T-22, T-140, TE-14011, T-14012, or TN14003, or an antibody that that specifically binds CXCR4. Additional CXCR4 antagonists are described, for example, in U.S. Patent Pub. No. 2014/0219952 and Debnath et al. (Theranostics, 2013; 3(1): 47-75), each of which is incorporated herein by reference in its entirety, and include TG-0054 (burixafor), AMD3465, NIBR1816, AMD070, and derivatives thereof. In some embodiments, a CXCR4 antagonist may be AMD3100 (plerixafor). AMD3100 is described in U.S. Pat. No. 5,583,131, which is incorporated by reference herein in its entirety.

In some embodiments, a CXCR7 antagonist can include, but is not limited to, an agent that inhibits binding of CXCL12 to CXCR7. In some embodiments, a CXCR7 antagonist can include, but is not limited to, CCX771, CCX754, or an antibody that specifically binds CXCR7 (e.g., interferes with dimerization of CXCR7).

The methods of the invention may further comprise delivering to the subject one or more additional therapeutic agents, wherein the additional therapeutic agents may be chemotherapeutic agents and/or a radiotherapeutic agents and/or an immunotherapeutic agents. The methods of the invention may further comprise surgery to remove some or all of the tumor and/or post-surgery disease reduction.

In some aspects, an immunotherapeutic agent may be a vaccine for inducing an immune response against a disease in a subject, an immune checkpoint inhibitor, a natural killer cell, a T-cell, and/or an antibody specific for diseased cells.

In some embodiments of the invention, when a vaccine is administered, it may be administered in more than one administration (e.g., two, three, four, or more administrations), which can be employed over a variety of time intervals (e.g., hourly, daily, weekly, monthly, etc.) to achieve therapeutic effects. Thus, for example, a vaccine may be administered in three doses at 0 months, 2 months and 6 months or at 0 months, 1 month and 6 months, or in two doses at 0 months and 6-12 months.

An immune checkpoint inhibitor may be any molecule that inhibits an immune checkpoint. Immune checkpoints are well known in the art and include, without limitation, PD-1, PD-L1, PD-L2, CTLA4, B7-H3, B7-H4, BTLA, IDO, KIR, LAG3, A2AR, TIM-3, and VISTA. In some embodiments, the inhibitor maybe an antibody against the immune checkpoint protein. In some embodiments, an immune checkpoint inhibitor may be an inhibitor of PD-1 or PD-L1, e.g., an antibody that specifically binds PD-1 or PD-L1. In some embodiments, an immune checkpoint inhibitor may be nivolumab, pembrolizumab, ipilimumab, durvalumab, or atezolizumab. In some embodiments, the immune checkpoint inhibitor is nivolumab. In some embodiments, the immune checkpoint inhibitor is pembrolizumab.

Inhibitors of CXCL12 signaling (e.g., CXCR4 and/or CXCR7 antagonists) and immune checkpoint inhibitors are known in the art, for example, see U.S. Publication Nos. 2016/0235779, 2016/0304607, 2015/0352208, and 2015/0216843 and International Publication Nos. WO 2017/019767, WO 2017/009842, and WO 2016/201425.

The immunotherapeutic agent may be an anti-cancer vaccine (also called cancer vaccine). Anti-cancer vaccines are vaccines that either treat existing cancer or prevent development of a cancer by stimulating an immune reaction to kill the cancer cells. In some embodiments, the anti-cancer vaccine treats existing cancer.

The anti-cancer vaccine may be any such vaccine having a therapeutic effect on one or more types of cancer. Many anti-cancer vaccines are currently known in the art. Such vaccines include, without limitation, dasiprotimut-T, Sipuleucel-T, talimogene laherparepvec, HSPPC-96 complex (Vitespen), L-BLP25, gplOO melanoma vaccine, and any other vaccine that stimulates an immune response to cancer cells when administered to a patient. Example MM vaccines include, but are not limited to, VIC-008, CRS-207 and WT1. An anti-cancer vaccine may be an engineered molecule that targets cancer cells and delivers an immunostimulatory agent. For example, the vaccine may be a fusion protein comprising an antigen targeting portion (e.g., an antibody such as a scFv) and an immunostimulatory portion (e.g., a stress protein such as a heat shock protein). See, for example, U.S. Pat. Nos. 7,749,501 and 8,143,387.

Immunotherapeutic agents can include natural killer cells, NK-92 cells, T cells, antibodies, and vaccines.

Natural killer (NK) cells are a class of lymphocytes that typically comprise approximately 10% of the lymphocytes in a human. NK cells provide an innate cellular immune response against tumor and infected (target) cells. NK cells, which are characterized as having a CD3⁻/CD56⁺ phenotype, display a variety of activating and inhibitory cell surface receptors. NK cell inhibitory receptors predominantly engage with major histocompatibility complex class I (“MHC-I”) proteins on the surface of a normal cell to prevent NK cell activation. The MHC-I molecules define cells as “belonging” to a particular individual. It is thought that NK cells can be activated only by cells on which these “self MHC-I molecules” are missing or defective, such as is often the case for tumor or virus-infected cells.

NK cells are triggered to exert a cytotoxic effect directly against a target cell upon binding or ligation of an activating NK cell receptor to the corresponding ligand on the target cell. The cytotoxic effect is mediated by secretion of a variety of cytokines by the NK cells, which in turn stimulate and recruit other immune system agents to act against the target. Activated NK cells also lyse target cells via the secretion of the enzymes perforin and granzyme, stimulation of apoptosis-initiating receptors, and other mechanisms.

NK cells have been evaluated as an immunotherapeutic agent in the treatment of certain cancers. NK cells used for this purpose may be autologous or non-autologous (i.e., from a donor).

In some embodiments, the NK cells used in the compositions and methods herein are autologous NK cells. In some embodiments, the NK cells used in the compositions and methods herein are non-autologous NK cells.

In some embodiments, the NK cells used in the compositions and methods herein are modified NK cells. NK cells may be modified by insertion of genes or RNA into the cells such that the cells express one or more proteins that are not expressed by wild type NK cells. In some embodiments, the NK cells may be modified to express a chimeric antigen receptor (CAR). In some embodiments, the CAR is specific for the cancer being targeted by the method or composition.

Non-limiting examples of modified NK cells can be found, for example, in Glienke, et al. 2015, Frontiers in Pharmacol. 6, article 21; PCT Patent Pub. Nos. WO 2013154760 and WO 2014055668; each of which is incorporated herein by reference in its entirety.

The NK-92 cell line was discovered in the blood of a subject suffering from a non-Hodgkins lymphoma. NK-92 cells lack the major inhibitory receptors that are displayed by normal NK cells, but retain a majority of the activating receptors. NK-92 cells are cytotoxic to a significantly broader spectrum of tumor and infected cell types than are NK cells and often exhibit higher levels of cytotoxicity toward these targets. However, NK-92 cells do not attack normal cells nor do they elicit an immune rejection response. In addition, NK-92 cells can be readily and stably grown and maintained in continuous cell culture and, thus, can be prepared in large quantities under c-GMP compliant quality control. This combination of characteristics has resulted in NK-92 being entered into presently on-going clinical trials for the treatment of multiple types of cancers.

NK-92 cells used in the compositions and methods described herein may be wild type (i e , unmodified) NK-92 cells or modified NK-92 cells. NK-92 cells can be modified by insertion of genes or RNA into the cells such that the cells express one or more proteins that are not expressed by wild type NK-92 cells. In some embodiments, NK-92 cells may be modified to express a chimeric antigen receptor (CAR) on the cell surface. In some embodiments, the CAR is specific for the cancer being targeted by the method or composition. In some embodiments, NK-92 cells may be modified to express an Fc receptor on the cell surface. In some embodiments, the NK-92 cell expressing the Fc receptor may mediate antibody-dependent cell-mediated cytotoxicity (ADCC). In some embodiments, the Fc receptor is CD 16. In some embodiments, NK-92 cells may be modified to express a cytokine (e.g., IL-2).

In one embodiment, the modified NK-92 cell is administered in combination with an antibody specific for the cancer to be treated. In one embodiment, the modified NK-92 cell administered in combination with the antibody is competent to mediate ADCC.

Non-limiting examples of modified NK-92 cells are described, for example, in U.S. Pat. Nos. 7,618,817 and 8,034,332; and U.S. Patent Pub. Nos. 2002/0068044 and 2008/0247990, each of which is incorporated herein by reference in its entirety. Non-limiting examples of CAR-modified NK-92 cells can be found, for example, in Glienke, et al. 2015, Frontiers in Pharmacol. 6, article 21; which is incorporated herein by reference in its entirety.

T cells are lymphocytes having T-cell receptor on the cell surface. T cells play a central role in cell-mediated immunity by tailoring the body's immune response to specific pathogens. T cells, especially modified T cells, have shown promise in reducing or eliminating tumors in clinical trials. Generally, such T cells are modified and/or undergo adoptive cell transfer (ACT). ACT and variants thereof are well known in the art. See, for example, U.S. Pat. Nos. 8,383,099 and 8,034,334, which are incorporated herein by reference in their entireties.

U.S. Patent App. Pub. Nos. 2014/0065096 and 2012/0321666, incorporated herein by reference in their entireties, describe methods and compositions for T cell or NK cell treatment of cancer. T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 2006/0121005, each of which is incorporated herein by reference in its entirety.

In some embodiments, T cells used in the compositions and methods herein may be autologous T cells (i.e., derived from the patient). In some embodiments, T cells used in the compositions and methods herein may be non-autologous (heterologous; e.g., from a donor or cell line) T cells. In some embodiments, the T cell may be a cell line derived from T cell(s) or cancerous/transformed T cell(s).

In some embodiments, a T cell used in the methods and compositions described herein may be a modified T cell. In some embodiments, the T cell may be modified to express a CAR on the surface of the T cell. In some embodiments, the CAR may be specific for the cancer being targeted by the method or composition. In some embodiments, a T cell may be modified to express a cell surface protein or cytokine. Exemplary, non-limiting examples of modified T cells are described in U.S. Pat. No. 8,906,682; PCT Patent Pub. Nos. WO 2013154760 and WO 2014055668; each of which is incorporated herein by reference in its entirety.

In some embodiments, the T cell may be a T cell line. Exemplary T cell lines include, but are not limited to, T-ALL cell lines, as described in U.S. Pat. No. 5,272,082, which is incorporated herein by reference in its entirety.

Immunotherapy may also refer to treatment with anti-tumor antibodies. That is, antibodies specific for a particular type of cancer (e.g., a cell surface protein expressed by the target cancer cells) may be administered to a patient having cancer. The antibodies may be monoclonal antibodies, polyclonal antibodies, chimeric antibodies, antibody fragments, human antibodies, humanized antibodies, or non-human antibodies (e.g., murine, goat, primate, etc.). The therapeutic antibody may be specific for any tumor-specific or tumor-associated antigen. See, e.g., Scott et al., Cancer Immunity 2012, 12: 14, which is incorporated herein by reference in its entirety.

In some embodiments, the immunotherapy agent may be an anti-cancer antibody. Non-limiting examples include trastuzumab (Herceptin®), bevacizumab (Avastin®), cetuximab (Erbitux®), panitumumab (Vectibix®), ipilimumab (Yervoy®), rituximab (Rituxan®), alemtuzumab (Campath®), ofatumumab (Arzerra®), gemtuzumab ozogamicin (Mylotarg®), brentuximab vedotin (Adcetris®), ⁹⁰Y-ibritumomab tiuxetan (Zevalin®), and ¹³¹I-tositumomab (Bexxar®).

Doses and administration protocols for immunotherapeutic drugs are well-known in the art. The skilled clinician can readily determine the proper dosing regimen to be used, based on factors including the immunotherapeutic agent(s) administered, type of cancer being treated, stage of the cancer, age and condition of the patient, patient size, location of the tumor, and the like. In some embodiments, immunotherapeutic agents may be administered at doses and schedules known in the art to be effective. In some embodiments, when combined with the methods of the present invention, immunotherapeutic agents may be administered at lower doses and/or with less frequency than typically used.

The chemotherapeutic agent may be any agent having a therapeutic effect on one or more types of cancer. Many chemotherapeutic agents are currently known in the art. Types of chemotherapy drugs include, by way of non-limiting example, alkylating agents, antimetabolites, anti-tumor antibiotics, topoisomerase inhibitors, mitotic inhibitors, corticosteroids, and the like.

Non-limiting examples of chemotherapeutic drugs include: nitrogen mustards, such as mechlorethamine (nitrogen mustard), chlorambucil, cyclophosphamide (Cytoxan®), ifosfamide, and melphalan); nitrosoureas, such as streptozocin, carmustine (BCNU), and lomustine; alkyl sulfonates, such as busulfan; triazines, such as dacarbazine (DTIC) and temozolomide (Temodar®); ethylenimines, such as thiotepa and altretamine (hexamethylmelamine); platinum drugs, such as cisplatin, carboplatin, and oxalaplatin; 5-fluorouracil (5-FU); 6-mercaptopurine (6-MP); capecitabine (Xeloda®); cytarabine (Ara-C®); floxuridine; fludarabine; gemcitabine (Gemzar®); hydroxyurea; methotrexate; pemetrexed (Alimta®); anthracyclines, such as daunorubicin, doxorubicin (Adriamycin®), epirubicin, idarubicin; actinomycin-D; bleomycin; mitomycin-C; mitoxantrone; topotecan; irinotecan (CPT-11); etoposide (VP-16); teniposide; mitoxantrone; taxanes: paclitaxel (Taxol®) and docetaxel (Taxotere®); epothilones: ixabepilone (Ixempra®); vinca alkaloids: vinblastine (Velban®), vincristine (Oncovin®), and vinorelbine (Navelbine®); estramustine (Emcyt®); prednisone; methylprednisolone (Solumedrol®); dexamethasone (Decadron®); L-asparaginase; and bortezomib (Velcade®).

Doses and administration protocols for chemotherapeutic drugs are well-known in the art. The skilled clinician can readily determine the proper dosing regimen to be used, based on factors including the chemotherapeutic agent(s) administered, type of cancer being treated, stage of the cancer, age and condition of the patient, patient size, location of the tumor, and the like. In some embodiments, chemotherapeutic agents may be administered at doses and schedules known in the art to be effective. In some embodiments, when combined with the methods of the present invention, chemotherapeutic agents may be administered at lower doses and/or with less frequency than typically used.

The radiotherapeutic agent may be any such agent having a therapeutic effect on one or more types of cancer. Many radiotherapeutic agents are currently known in the art. Types of radiotherapeutic drugs include, by way of non-limiting example, X-rays, gamma rays, and charged particles. In some embodiments, the radiotherapeutic agent may be delivered by a machine outside of the body (external-beam radiation therapy). In some embodiments, the radiotherapeutic agent may be placed in the body near the tumor/cancer cells (brachytherapy) or is a systemic radiation therapy.

External-beam radiation therapy may be administered by any means. Exemplary, non-limiting types of external-beam radiation therapy include linear accelerator-administered radiation therapy, 3-dimensional conformal radiation therapy (3D-CRT), intensity-modulated radiation therapy (IMRT), image-guided radiation therapy (IGRT), tomotherapy, stereotactic radiosurgery, photon therapy, stereotactic body radiation therapy, proton beam therapy, and electron beam therapy.

Internal radiation therapy (brachytherapy) may be by any technique or agent. Exemplary, non-limiting types of internal radiation therapy include any radioactive agents that can be placed proximal to or within the tumor, such as radium-226 (Ra-226), cobalt-60 (Co-60), cesium-137 (Cs-137), cesium-131, iridium-192 (Ir-192), gold-198 (Au-198), iodine-125 (I-125), palladium-103, yttrium-90, etc. Such agents may be administered by seeds, needles, or any other route of administration, and may be temporary or permanent.

Systemic radiation therapy may be by any technique or agent. Exemplary, non-limiting types of systemic radiation therapy include radioactive iodine, ibritumomab tiuxetan (Zevalin®), tositumomab and iodine-131 tositumomab (Bexxar®), samarium-153-lexidronam (Quadramet®), strontium-89 chloride (Metastron®), metaiodobenzylguanidine, lutetium-177, yttrium-90, strontium-89, and the like.

In some embodiments, a radiosensitizing agent may also be administered to the patient. Radiosensitizing agents increase the damaging effect of radiation on cancer cells.

Doses and administration protocols for radiotherapy agents are well-known in the art. The skilled clinician can readily determine the proper dosing regimen to be used, based on factors including the agent(s) administered, type of cancer being treated, stage of the cancer, location of the tumor, age and condition of the patient, patient size, and the like. In some embodiments, radiotherapeutic agents may be administered at doses and schedules known in the art to be effective. In some embodiments, when combined with the methods of the present invention, radiotherapeutic agents may be administered at lower doses and/or with less frequency than typically used.

An inhibitor of CXCL12 signaling (e.g., a CXCR4 and/or CXCR7 antagonist) and an optional additional therapeutic agent may be delivered to the subject in any manner or pattern that is effective. In some embodiments, the inhibitor of CXCL12 signaling and additional therapeutic agent may be delivered to the subject in the same composition. In other embodiments, the inhibitor of CXCL12 signaling, and the additional therapeutic agent may be delivered to the subject in separate compositions. The two agents may be delivered to the subject simultaneously. The two agents (e.g., inhibitor of CXCL12 signaling, and additional therapeutic agent) may be delivered to the subject sequentially and the sequence may be repeated as necessary, e.g., repeated 1, 2, 3, 4, 5, 6, 7, 8, 9, or 10 time or more.

In some embodiments, the two agents may be delivered in the same pattern and/or schedule. In other embodiments, the two agents may be delivered in a different pattern and/or schedule. In some embodiments, the additional therapeutic agent may be an immunotherapeutic agent that may be administered to the subject for a sufficient amount of time to stimulate the immune system and then stopped. For example, the immunotherapeutic agent may be administered for just a few doses, e.g., 10 doses or less, e.g., 10, 9, 8, 7, 6, 5, 4, 3, 2, or 1 dose, in a periodic fashion, e.g., once every week, 2 weeks, 3 weeks, 4 weeks, 5 weeks, 6 weeks, or more. In some embodiments, the CXCR4 antagonist and/or CXCR7 antagonist may be administered for a longer period of time than the additional therapeutic agent, e.g., until the disease (e.g., cancer or infection) has been successfully treated. The inhibitor of CXCL12 signaling also may be administered more frequently than the additional therapeutic agent, e.g., once every 3 hours, 4 hours, 6 hours, 12 hours, day, 2 days, 3 days, 4 days, 5, days, 6, days, week, or more.

In some embodiments, the inhibitor of CXCL12 signaling (e.g., CXCR4 and/or CXCR7 antagonist) of the invention is administered directly to a subject. Generally, the antagonist of the invention will be suspended in a pharmaceutically-acceptable carrier (e.g., physiological saline) and administered orally or by intravenous infusion, or administered subcutaneously, intramuscularly, intrathecally, intraperitoneally, intrarectally, intravaginally, intranasally, intragastrically, intratracheally, intrapulmonarily or transdermally. In some embodiments, the intratracheal or intrapulmonary delivery may be accomplished using a standard nebulizer, jet nebulizer, wire mesh nebulizer, dry powder inhaler, or metered dose inhaler. They may be delivered directly to the site of the disease or disorder, such as the skin, the cervix, the head, the neck, or directly into a tumor or lesion.

In some embodiments, the inhibitor of CXCL12 signaling may be administered proximal to (e.g., near or within the same body cavity as) the tumor, e.g., into the peritoneal or pleural cavity, or topically, e.g., to the skin or a mucosal surface, e.g., to the cervix. In some embodiments, the inhibitor may be administered directly into the tumor or into a blood vessel feeding the tumor. In some embodiments, the inhibitor may be administered systemically. In some embodiments, the inhibitor may be administered by microcatheter, or an implanted device, or an implanted dosage form.

In some embodiments, the inhibitor of CXCL12 signaling (e.g., CXCR4 and/or CXCR7 antagonist) may be administered in a continuous manner for a defined period. In some embodiments, the inhibitor may be administered in a pulsatile manner. For example, the inhibitor may be administered intermittently over a period of time. The inhibitor may be administered in the same or different patterns and for the same or different lengths of time.

The dosage required depends on the choice of the route of administration; the nature of the formulation; the nature of the patient's illness; the subject's size, weight, surface area, age, and sex; other drugs being administered; and the judgment of the attending physician. Wide variations in the needed dosage are to be expected in view of the variety of molecules available and the differing efficiencies of various routes of administration. For example, oral administration would be expected to require higher dosages than administration by i.v. injection. Variations in these dosage levels can be adjusted using standard empirical routines for optimization as is well understood in the art. Administrations can be single or multiple (e.g., 2-, 3-, 4-, 6-, 8-, 10-, 20-, 50-, 100-, 150-, or more fold). Encapsulation of an inhibitor of CXCL12 signaling (e.g., CXCR4 and/or CXCR7 antagonist) in a suitable delivery vehicle (e.g., polymeric microparticles or nanoparticles, slow release polymeric gels, or implantable devices) may increase the efficiency of delivery, particularly for oral delivery or delivery into or nearby the location of a tumor, e.g., the peritoneal or pleural cavity.

Generally, the dose of an inhibitor of CXCL12 signaling (e.g., CXCR4 and/or CXCR7 antagonist) of the present invention may be from about 0.01 mg/kg body weight per day to about 500 mg/kg per day, e.g., about 0.1 mg/kg body weight per day to about 20 mg/kg per day, about 0.1 mg/kg body weight per day to about 30 mg/kg per day, about 0.1 mg/kg body weight per day to about 40 mg/kg per day, about 0.1 mg/kg body weight per day to about 50 mg/kg per day, about 0.1 mg/kg body weight per day to about 75 mg/kg per day, about 0.1 mg/kg body weight per day to about 100 mg/kg per day, about 1 mg/kg per day to about 100 mg/kg per day, about 1 mg/kg per day to about 150 mg/kg per day, about 1 mg/kg per day to about 200 mg/kg per day, about 1 mg/kg per day to about 250 mg/kg per day, about 1 mg/kg per day to about 300 mg/kg per day, about 1 mg/kg per day to about 350 mg/kg per day, about 1 mg/kg per day to about 400 mg/kg per day, about 1 mg/kg per day to about 450 mg/kg per day, about 1 mg/kg per day to about 500 mg/kg per day, about 10 mg/kg per day to about 200 mg/kg per day, about 10 mg/kg per day to about 250 mg/kg per day, about 10 mg/kg per day to about 300 mg/kg per day, about 10 mg/kg per day to about 350 mg/kg per day, about 10 mg/kg per day to about 400 mg/kg per day, about 10 mg/kg per day to about 450 mg/kg per day, about 10 mg/kg per day to about 500 mg/kg per day, about 50 mg/kg per day to about 200 mg/kg per day, about 50 mg/kg per day to about 250 mg/kg per day, about 50 mg/kg per day to about 300 mg/kg per day, about 50 mg/kg per day to about 350 mg/kg per day, about 50 mg/kg per day to about 400 mg/kg per day, about 50 mg/kg per day to about 450 mg/kg per day, about 50 mg/kg per day to about 500 mg/kg per day, inclusive of all values and ranges therebetween, including endpoints. In some embodiments, the dose may be from about 0.1 mg/kg to about 50 mg/kg per day. In some embodiments, the dose may be from about 0.1 mg/kg to about 40 mg/kg per day. In some embodiments, the dose may be from about 0.1 mg/kg to about 30 mg/kg per day. In some embodiments, the dose may be from about 0.1 mg/kg to about 20 mg/kg per day. In some embodiments, the dose does not exceed about 50 mg per day.

In some embodiments, the dose may be from about 0.5 mg/kg per week to about 350 mg/kg per week, inclusive of all values and ranges therebetween, including endpoints. In one embodiment, the dose is about 0.5 mg/kg per week. In one embodiment, the dose is about 1 mg/kg per week. In one embodiment, the dose is about 2 mg/kg per week. In one embodiment, the dose is about 5 mg/kg per week. In one embodiment, the dose is about 10 mg/kg per week. In one embodiment, the dose is about 20 mg/kg per week. In one embodiment, the dose is about 30 mg/kg per week. In one embodiment, the dose is about 40 mg/kg per week. In one embodiment, the dose is about 50 mg/kg per week. In one embodiment, the dose is about 60 mg/kg per week. In one embodiment, the dose is about 70 mg/kg per week. In one embodiment, the dose is about 80 mg/kg per week. In one embodiment, the dose is about 90 mg/kg per week. In one embodiment, the dose is about 100 mg/kg per week. In one embodiment, the dose is about 110 mg/kg per week. In one embodiment, the dose is about 120 mg/kg per week. In one embodiment, the dose is about 130 mg/kg per week. In one embodiment, the dose is about 140 mg/kg per week. In one embodiment, the dose is about 150 mg/kg per week. In one embodiment, the dose is about 160 mg/kg per week. In one embodiment, the dose is about 170 mg/kg per week. In one embodiment, the dose is about 180 mg/kg per week. In one embodiment, the dose is about 190 mg/kg per week. In one embodiment, the dose is about 200 mg/kg per week. In one embodiment, the dose is about 210 mg/kg per week. In one embodiment, the dose about 220 mg/kg per week. In one embodiment, the dose is about 230 mg/kg per week. In one embodiment, the dose is about 240 mg/kg per week. In one embodiment, the dose is about 250 mg/kg per week. In one embodiment, the dose is about 260 mg/kg per week. In one embodiment, the dose is about 270 mg/kg per week. In one embodiment, the dose is about 280 mg/kg per week. In one embodiment, the dose is about 290 mg/kg per week. In one embodiment, the dose is about 300 mg/kg per week. In one embodiment, the dose is about 310 mg/kg per week. In one embodiment, the dose is about 320 mg/kg per week. In one embodiment, the dose is about 330 mg/kg per week. In one embodiment, the dose is about 340 mg/kg per week. In one embodiment, the dose is about 350 mg/kg per week.

In some embodiments of the invention, administration of the inhibitor of CXCL12 signaling (e.g., CXCR4 and/or CXCR7 antagonist) may be pulsatile. In some embodiments, an amount of the inhibitor of CXCL12 signaling may be administered every 1 hour to every 24 hours, for example every 1 hour, 2 hours, 3 hours, 4 hours, 5 hours, 6 hours, 7 hours, 8 hours, 9 hours, 10 hours, 11 hours, 12 hours, 13 hours, 14 hours 15 hours, 16 hours, 17 hours, 18 hours, 19 hours, 20 hours, 21 hours, 22 hours, 23 hours, or 24 hours. In some embodiments, an amount of the inhibitor of CXCL12 signaling may be administered every 1 day, 2 days, 3 days, 4 days, 5 days, 6 days, 7 days, 8 days, 9 days, or 10 days.

In some embodiments, the administration of the inhibitor of CXCL12 signaling may be of indefinite duration, to be determined by the managing physician, and only terminated when the disease is judged to be either cured or in remission.

In some embodiments, when inhibitors of CXCL12 signaling are provided with an additional therapeutic agent (e.g., immunotherapeutic agent, immune checkpoint inhibitor, anti-cancer vaccine (e.g., MM vaccine); chemotherapeutic agent; radiotherapeutic agent), the administration of the inhibitor of CXCL12 signaling and the additional therapeutic agent may be alternated. In one embodiment, administration of the inhibitor of CXCL12 signaling and the additional therapeutic agent may be alternated until the condition of the patient improves. Improvement includes, without limitation, reduction in size of the tumor and/or metastases thereof, elimination of the tumor and/or metastases thereof, remission of the cancer, and/or attenuation of at least one symptom of the cancer and/or infectious disease.

According to some embodiments, an inhibitor of CXCL12 signaling (e.g., CXCR4 and/or CXCR7 antagonist) may be targeted to specific cells or tissues in vivo. Targeting delivery vehicles, including liposomes and targeted systems are known in the art. For example, a liposome or particle can be directed to a particular target cell or tissue, e.g., a cancer cell, tumor, or lesion, by using a targeting agent, such as an antibody, soluble receptor or ligand, incorporated with the liposome or particle, to target a particular cell or tissue to which the targeting molecule can bind. Targeting liposomes are described, for example, in Ho et al., Biochemistry 25:5500 (1986); Ho et al., J. Biol. Chem. 262:13979 (1987); Ho et al., J. Biol. Chem. 262:13973 (1987); and U.S. Pat. No. 4,957,735 to Huang et al., each of which is incorporated herein by reference in its entirety).

In some embodiments, an inhibitor of CXCL12 signaling (e.g., CXCR4 and/or CXCR7 antagonist) and an additional therapeutic agent may be administered by the same route. In other embodiments, the inhibitor and the additional therapeutic agent may be administered by different routes, e.g., by the route most suitable for each agent. For example, the additional therapeutic agent may be administered systemically (e.g., intravenously) and the inhibitor may be administered locally (e.g., directly into a tumor or into a body cavity containing the tumor) or the inhibitor may be administered systemically and the additional therapeutic agent may be administered locally. In some embodiments, an immune checkpoint inhibitor may be administered by intravenous infusion and the CXCL12 signaling inhibitor may be administered by subcutaneous injection or by subcutaneous pump to a local tumor site or for systemic delivery.

As a further aspect, the invention provides pharmaceutical formulations and methods of administering the same to achieve any of the therapeutic effects (e.g., treatment of malignant mesothelioma) discussed above. The pharmaceutical formulation may comprise any of the reagents discussed above in a pharmaceutically acceptable carrier.

By “pharmaceutically acceptable” it is meant a material that is not biologically or otherwise undesirable, i.e., the material can be administered to a subject without causing any undesirable biological effects such as toxicity.

The formulations of the invention can optionally comprise medicinal agents, pharmaceutical agents, carriers, adjuvants, dispersing agents, diluents, and the like.

The agents of the invention can be formulated for administration in a pharmaceutical carrier in accordance with known techniques. See, e.g., Remington, The Science And Practice of Pharmacy (9^{th} Ed. 1995). In the manufacture of a pharmaceutical formulation according to the invention, the agent (including the physiologically acceptable salts thereof) is typically admixed with, inter alia, an acceptable carrier. The carrier can be a solid or a liquid, or both, and is preferably formulated with the agent as a unit-dose formulation, for example, a tablet, which can contain from 0.01 or 0.5% to 95% or 99% by weight of the agent. One or more agents can be incorporated in the formulations of the invention, which can be prepared by any of the well-known techniques of pharmacy.

A further aspect of the invention is a method of treating subjects in vivo, comprising administering to a subject a pharmaceutical composition comprising an inhibitor of CXCL12 signaling (e.g., CXCR4 and/or CXCR7 antagonist) in a pharmaceutically acceptable carrier, wherein the pharmaceutical composition is administered in a therapeutically effective amount. Administration of the inhibitor of the present invention to a human subject or an animal in need thereof can be by any means known in the art for administering compounds.

The formulations of the invention include those suitable for oral, rectal, perianal, topical, buccal (e.g., sub-lingual), vaginal, parenteral (e.g., subcutaneous, intramuscular including skeletal muscle, cardiac muscle, diaphragm muscle and smooth muscle, intradermal, intravenous, intraperitoneal), topical (i.e., both skin and mucosal surfaces, including airway surfaces), intranasal, transdermal, intraarticular, intrathecal, and inhalation administration, administration to the liver by intraportal delivery, as well as direct organ injection (e.g., into the liver, into the brain for delivery to the central nervous system, into the pancreas, or into a tumor or the tissue surrounding a tumor). The most suitable route in any given case will depend on the nature and severity of the condition being treated and on the nature of the particular treatment being administered.

For injection, the carrier will typically be a liquid, such as sterile pyrogen-free water, pyrogen-free phosphate-buffered saline solution, bacteriostatic water, or Cremophor EL[R] (BASF, Parsippany, N.J.). For other methods of administration, the carrier can be either solid or liquid.

For oral administration, an inhibitor of CXCL12 signaling (e.g., CXCR4 and/or CXCR7 antagonist) may be administered in solid dosage forms, such as capsules, tablets, and powders, or in liquid dosage forms, such as elixirs, syrups, and suspensions. The inhibitor of CXCL12 signaling may be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate and the like. Examples of additional inactive ingredients that can be added to provide desirable color, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for buccal (sub-lingual) administration include lozenges comprising the inhibitor of CXCL12 signaling in a flavored base, usually sucrose and acacia or tragacanth; and pastilles comprising the antagonist in an inert base such as gelatin and glycerin or sucrose and acacia.

Formulations of the present invention suitable for parenteral administration comprise sterile aqueous and non-aqueous injection solutions of the inhibitor of CXCL12 signaling, which preparations are preferably isotonic with the blood of the intended recipient. These preparations can contain anti-oxidants, buffers, bacteriostats and solutes which render the formulation isotonic with the blood of the intended recipient. Aqueous and non-aqueous sterile suspensions can include suspending agents and thickening agents. The formulations can be presented in unit/dose or multi-dose containers, for example sealed ampoules and vials, and can be stored in a freeze-dried (lyophilized) condition requiring only the addition of the sterile liquid carrier, for example, saline or water-for-injection immediately prior to use.

Extemporaneous injection solutions and suspensions can be prepared from sterile powders, granules and tablets of the kind previously described. For example, in one aspect of the present invention, there is provided an injectable, stable, sterile composition comprising an inhibitor of CXCL12 signaling (e.g., CXCR4 and/or CXCR7 antagonist) in a unit dosage form in a sealed container. The inhibitor of CXCL12 signaling and/or the additional therapeutic agent may be provided in the form of a lyophilizate which is capable of being reconstituted with a suitable pharmaceutically acceptable carrier to form a liquid composition suitable for injection thereof into a subject. The unit dosage form may comprise from about 0.01 mg to about 10 grams of the inhibitor and/or the additional therapeutic agent. When the inhibitor and/or the additional therapeutic agent are substantially water-insoluble, a sufficient amount of emulsifying agent which is pharmaceutically acceptable can be employed in sufficient quantity to emulsify the inhibitor and/or agent in an aqueous carrier. One such useful emulsifying agent is phosphatidyl choline.

Formulations suitable for rectal administration are preferably presented as unit dose suppositories. These can be prepared by admixing the inhibitor of CXCL12 signaling and/or agent with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which can be used include petroleum jelly, lanoline, polyethylene glycols, alcohols, transdermal enhancers, and combinations of two or more thereof.

Formulations suitable for transdermal administration can be presented as discrete patches adapted to remain in intimate contact with the epidermis of the recipient for a prolonged period of time. Formulations suitable for transdermal administration can also be delivered by iontophoresis (see, for example, Tyle, Pharm. Res. 3:318 (1986)) and typically take the form of an optionally buffered aqueous solution of the polypeptides. Suitable formulations comprise citrate or bis/tris buffer (pH 6) or ethanol/water and contain from 0.1 to 0.2M of the compound.

The an inhibitor of CXCL12 signaling (e.g., CXCR4 and/or CXCR7 antagonist) and/or additional therapeutic agent can alternatively be formulated for nasal administration or otherwise administered to the lungs of a subject by any suitable means, e.g., administered by an aerosol suspension of respirable particles comprising the inhibitor and/or agent, which the subject inhales. The respirable particles can be liquid or solid. The term “aerosol” includes any gas-borne suspended phase, which is capable of being inhaled into the bronchioles or nasal passages. Specifically, aerosol includes a gas-borne suspension of droplets, as can be produced in a metered dose inhaler or nebulizer, or in a mist sprayer. Aerosol also includes a dry powder composition suspended in air or other carrier gas, which can be delivered by insufflation from an inhaler device, for example. See Ganderton & Jones, Drug Delivery to the Respiratory Tract, Ellis Horwood (1987); Gonda (1990) Critical Reviews in Therapeutic Drug Carrier Systems 6:273-313; and Raeburn et al., J. Pharmacol. Toxicol. Meth. 27:143 (1992). Aerosols of liquid particles comprising the inhibitor and/or agent may be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the inhibitor and/or agent can likewise be produced with any solid particulate medicament aerosol generator, by techniques known in the pharmaceutical art.

Alternatively, one can administer the CXCL12 signaling inhibitor and/or additional therapeutic agent in a local rather than systemic manner, for example, in a depot or sustained-release formulation.

Further, the present invention provides liposomal formulations of an inhibitor of CXCL12 signaling (e.g., CXCR4 and/or CXCR7 antagonist) and/or additional therapeutic agent disclosed herein and salts thereof. The technology for forming liposomal suspensions is well known in the art. When the inhibitor and/or agent or salt thereof is an aqueous-soluble salt, using conventional liposome technology, the same can be incorporated into lipid vesicles. In such an instance, due to the water solubility of the inhibitor/agent or salt, the inhibitor/agent or salt thereof will be substantially entrained within the hydrophilic center or core of the liposomes. The lipid layer employed can be of any conventional composition and can either contain cholesterol or can be cholesterol-free. When the inhibitor/agent or salt of interest is water-insoluble, again employing conventional liposome formation technology, the salt can be substantially entrained within the hydrophobic lipid bilayer which forms the structure of the liposome. In either instance, the liposomes which are produced can be reduced in size, as through the use of standard sonication and homogenization techniques.

The liposomal formulations containing an inhibitor of CXCL12 signaling (e.g., CXCR4 and/or CXCR7 antagonist) and/or additional therapeutic agent disclosed herein or salts thereof, can be lyophilized to produce a lyophilizate which can be reconstituted with a pharmaceutically acceptable carrier, such as water, to regenerate a liposomal suspension.

In the case of water-insoluble inhibitors of CXCL12 signaling, a pharmaceutical composition can be prepared containing the water-insoluble antagonist, such as for example, in an aqueous base emulsion. In such an instance, the composition will contain a sufficient amount of pharmaceutically acceptable emulsifying agent to emulsify the desired amount of the inhibitor. Particularly useful emulsifying agents include phosphatidyl cholines and lecithin.

In some embodiments, the inhibitor of CXCL12 signaling (e.g., CXCR4 and/or CXCR7 antagonist) and/or additional therapeutic agent may be administered to a subject in a therapeutically effective amount, as that term is defined above. Dosages of pharmaceutically active antagonists/agents can be determined by methods known in the art, see, e.g., Remington's Pharmaceutical Sciences (Maack Publishing Co., Easton, Pa.). The therapeutically effective dosage of any specific CXCL12 signaling inhibitor or other therapeutic agent will vary somewhat from inhibitor to inhibitor, agent to agent, and patient to patient, and will depend upon the condition of the patient and the route of delivery. As a general proposition, a dosage from about 0.1 to about 50 mg/kg will have therapeutic efficacy, with all weights being calculated based upon the weight of the inhibitor/agent, including the cases where a salt is employed. Toxicity concerns at the higher level can restrict intravenous dosages to a lower level such as up to about 10 mg/kg, with all weights being calculated based upon the weight of the antagonist, including the cases where a salt is employed. A dosage from about 10 mg/kg to about 50 mg/kg can be employed for oral administration. Typically, a dosage from about 0.5 mg/kg to 5 mg/kg can be employed for intramuscular injection. Particular dosages are about 1 μmol/kg to 50 μmol/kg, and more particularly to about 22 μmol/kg and to 33 μmol/kg of the inhibitor of CXCL12 signaling and/or additional therapeutic agent for intravenous or oral administration, respectively.

Other delivery systems can include time-release, delayed release or sustained release delivery systems. Such systems can avoid repeated administrations of an inhibitor of CXCL12 signaling (e.g., CXCR4 and/or CXCR7 antagonist) and/or additional therapeutic agent, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono- di- and tri-glycerides; hydrogel release systems; sylastic systems; peptide based systems; wax coatings; compressed tablets using conventional binders and excipients; partially fused implants; and the like.

In some embodiments, an inhibitor of CXCL12 signaling (e.g., CXCR4 and/or CXCR7 antagonist) and/or additional therapeutic agent may be administered in a time-release, delayed release or sustained release delivery system. In some embodiments, the time-release, delayed release or sustained release delivery system comprising the inhibitor and/or agent may be inserted directly into the tumor. In some embodiments, the time-release, delayed release or sustained release delivery system comprising the inhibitor and/or agent may be implanted in the patient proximal to the tumor. Additional implantable formulations are described, for example, in U.S. Patent App. Pub. No. 2008/0300165, which is incorporated herein by reference in its entirety.

In addition, important embodiments of the invention include pump-based hardware delivery systems, some of which are adapted for implantation. Such implantable pumps include controlled-release microchips. A preferred controlled-release microchip is described in Santini, J T Jr. et al., Nature, 1999, 397:335-338, the contents of which are expressly incorporated herein by reference.

When administered, the pharmaceutical preparations of the invention are applied in pharmaceutically-acceptable amounts and in pharmaceutically-acceptable compositions. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

The present invention finds use in veterinary and medical applications. Suitable subjects include both birds and mammals, with mammals being preferred. The term “bird” as used herein includes, but is not limited to, chickens, ducks, geese, quail, turkeys, and pheasants. The term “mammal” as used herein includes, but is not limited to, humans, cattle, sheep, goats, horses, cats, dogs, lagomorphs, etc. Human subjects include neonates, infants, juveniles, and adults.

Another aspect of the invention relates to a composition comprising a inhibitor of CXCL12 signaling (e.g., CXCR4 and/or CXCR7 antagonist) and a vaccine for inducing an immune response against a disease (e.g., MM) in a subject. Example MM vaccines include, but are not limited to, VIC-008, CRS-207 and WT1.

Some aspects of the invention relate to a kit of parts comprising a container comprising a CXCR4 antagonist and/or CXCR7 antagonist. A further aspect of the invention relates to a kit of parts comprising a first container comprising a CXCR4 antagonist and/or CXCR7 antagonist and a second container comprising an additional therapeutic agent (for example, an anticancer vaccine).

The CXCR4 antagonist and/or CXCR7 antagonist and additional therapeutic agent in the composition or the kit of parts may be any of the agents described above.

A container may be, without limitation, a vial containing a single dose or multiple doses of the CXCR4 antagonist and/or CXCR7 antagonist or vaccine or a prefilled syringe containing the antagonist or vaccine.

In one embodiment, the composition or kit of parts may further comprise instructions in a readable medium for dosing and/or administration of the CXCR4 antagonist and/or CXCR7 antagonist and additional therapeutic agent. The term “readable medium” as used herein refers to a representation of data that can be read, for example, by a human or by a machine. Non-limiting examples of human-readable formats include pamphlets, inserts, or other written forms. Non-limiting examples of machine-readable formats include any mechanism that provides (i.e., stores and/or transmits) information in a form readable by a machine (e.g., a computer, tablet, and/or smartphone). For example, a machine-readable medium includes read-only memory (ROM); random access memory (RAM); magnetic disk storage media; optical storage media; and flash memory devices. In one embodiment, the machine-readable medium is a CD-ROM. In one embodiment, the machine-readable medium is a USB drive. In one embodiment, the machine-readable medium is a Quick Response Code (QR Code) or other matrix barcode.

The present invention is more particularly described in the following examples that are intended as illustrative only since numerous modifications and variations therein will be apparent to those skilled in the art.

### EXAMPLES

The overall objective response rate of locally recurrent and/or metastatic (R/M) head and neck squamous cell carcinoma (HNSCC) patients to anti-PD1 monotherapy is 18%. While these response rates are promising, there is an opportunity to increase the clinical impact of pembrolizumab with novel combinatorial strategies. Immune checkpoint blockade therapy potentiates the activity of existing CD8+ T cells and, ultimately, clinical efficacy can be limited by the number of infiltrating CD8+ T cells within the tumor microenvironment. Head and neck cancer patients who failed anti-PD-1 monotherapy may have failed due to a paucity of CD8+ T cells present within the tumor microenvironment.

CXCL12 is a chemoattractant for CD8+ T cells and our group has reported that high levels of CXCL12 paradoxically can elicit an immunosuppressive microenvironment with decreased CD8+ T cell infiltration through increased binding of CXCR4 expressed on the T cells (resulting in chemorepulsion) and increased recruitment of immunosuppressive CD4+FoxP3+ T regulatory (Treg) cells (J Immunol 2006; 176:2902-2914; Cancer Res 2011 71(16):5522-34). The present study is directed to targeting the CXCL12:CXCR4 axis to determine if intratumoral CD8+ T cell recruitment may be increased and, thus, increase the clinical response rates to anti-PD-1 therapy.

Plerixafor (AMD3100), a small molecule, is approved by the US Food and Drug Administration (FDA) for mobilizing hematopoietic stem cells from the bone marrow to the blood for transplantation in cancer and is a highly specific antagonist of CXCR4. Administration of AMD3100 can rapidly mobilize all major subsets of mature leukocytes into the blood. A phase I clinical trial demonstrated the safety of long-term, low-dose treatment with AMD3100 at a dose of 0.01-0.02 mg/kg (4-8% of the FDA-approved dose) administered subcutaneously twice daily for 6 months (Blood 2014; 123(15):2308-16).

### Example 1

### Materials and Methods

### Reagents

The fusion proteins were constructed as described previously (13) and expressed by WuXi Biologics (Shanghai, China) in CHO cells and provided at a purity of above 95% by HPLC and an endotoxin level of less than 1.0 EU/mg. AMD3100 was purchased from Abeam (#ab120718).

### Tumor Cells

40 L and AE17 mouse mesothelioma cell lines were kind gifts from Dr. Agnes Kane in the Department of Pathology and Laboratory Medicine at Brown University. Cells were cultured at 37° C. in DMEM supplemented with 1% L-glutamine, 1% penicillin-streptomycin, and 10% fetal bovine serum.

### Animal Models

Five-week-old female C57BL/6 mice were obtained from the Jackson Laboratory and maintained in the gnotobiotic animal facility of Massachusetts General Hospital (MGH) in compliance with institutional guidelines and policies. After one week acclimatization, tumors were initiated with 4×10⁶ 40 L cells or 2×10⁶ AE17 cells per mouse administered intraperitoneally (i.p.). A subset of the mice from each group were euthanized with i.p. administration of Ketamine (9 mg/ml in saline) and Xylazine (0.9 mg/ml in saline) 7 days after the last treatment, and samples harvested for immune profiling of tumors, lymph nodes and spleens. The remaining animals in each group were monitored for survival. For survival studies, the mice were observed daily after inoculation of tumor cells. Tumor generation was consistently first evident via the appearance of abdominal distension secondary to malignant ascites, and tumor-bearing mice were euthanized at the endpoint when there were signs of distress, including fur ruffling, rapid respiratory rate, hunched posture, reduced activity, and progressive ascites formation.

Splenocytes from transgenic T-Red/FoxP3 GFP mice were used as a source of fluorescently tagged T_{reg} cells by cell sorting as described below. T-Red/FoxP3 GFP mice are a fully backcrossed C57BL/6 line of transgenic mice that were produced by crossing T-red mice with FoxP3 GFP mice. T-red mice express dsRedII under the control of the CD4 promoter modified to lack the negative control element thereby allowing expression in both CD4⁺ and CD8⁺ T cells. In FoxP3 GFP mice, GFP is expressed from under control of the FoxP3 promoter with internal deletions to FoxP3 to prevent over expression. All animal studies were approved by the Institutional Animal Care and Use Committee of MGH.

### Treatment

Beginning seven days after tumor inoculation, treatments were administrated by i.p. injection once a week for four successive weeks. VIC-008 was administrated i.p. at 20 μg in 100 μl of saline per mouse once a week and AMD3100 was given once a week by i.p. injection at 1 mg/kg of body weight in 100 μl of saline.

### Immune Profiling by Flow Cytometry

Tumors were mechanically disaggregated using sterile razor blades and digested at 37° C. for 2 hours in RPMI 1640 with collagenase type IV for 40 L tumors or type I for AE17 tumors (2 mg/ml, Sigma), DNase (0.1 mg/ml, Sigma), hyaluronidase (0.1 mg/ml, Sigma), and BSA (2 mg/ml, Sigma). Cell suspensions were passed through 100 μm filters to remove aggregates. Lymph nodes and spleens were mashed and filtered through 40 μm strainers. Cells were washed with staining buffer (#420201, Biolegend) and stained with the conjugated antibodies for surface markers. Total live cells were determined by LIVE/DEAD® staining (ThermoFisher, #L23105).

For intracellular cytokine detection, after staining of surface markers, cells were fixed and permeabilized with fixation/permeabilization reagents from BioLegend (#424401) or eBioscience (#00-5521-00) and stained with the conjugated antibodies for intracellular markers.

Conjugated antibodies from eBioscience were as follows: CD40L (clone MR1), and PD-1 (clone J43). The following conjugated antibodies were purchased from BioLegend: CD3 (clone 17A2), CD4 (clone GK1.5), CD8a (clone 53-6.7), Foxp3 (clone MF-14), IL-2 (clone JES6-5H4), and IFN-γ (clone XMG1.2). CD25 (clone PC61) antibody was from BD Biosciences.

Flow cytometric analyses were performed using BD LSRFortessa X-20 (BD Biosciences). Gating strategies were determined by the Fluorescence Minus One. Flow data were analyzed by FlowJo V10 (TreeStar).

### Ex-Vivo Culturing of Splenocytes and Cytokine Detection

The splenocytes were harvested from mashed spleens, filtered through 40 μm cell strainers and treated with red blood cell lysis buffer. 2×10⁶ splenocytes were placed per well in 24-well plates in RPMI 1640 medium supplemented with L-glutamine and stimulated with 2 μg/ml of recombinant mouse mesothelin (BioLegend, #594006) for 72 hours. Brefeldin A and Monesin (BioLegend, #420601 and #420701) were added into the culture medium during the last five hours. Splenocytes were then harvested and intracellular cytokine staining performed using the corresponding antibodies for cytometric analysis.

### In Vitro Reprogramming of T_{reg} Cells

rGH-ffLuc-eGFP transgenic mice express Green Fluorescent Protein (GFP) in Foxp3⁺ T_{reg} cells. Spleens were collected from these mice and mashed and filtered through 40 μm strainers. Cells expressing GFP-Foxp3 from CD4⁺ splenocytes were sorted on a FACSAria (BD Biosciences) and then exposed to AMD3100 (5 μg/ml) in the presence or absence of anti-CD3/CD28 antibody (1 μg/ml) for 24 hours. Brefeldin A and Monesin (BioLegend, #420601 and #420701) were added into the culture medium during the last five hours. The cells were then harvested and stained with the conjugated antibodies specific for CD3, CD4, CD25, Foxp3, IL-2 and CD40L, and analyzed by flow cytometry.

### Statistical Analyses

P values were calculated by GraphPad Prism 6. Unless described otherwise, the P values for comparison among groups were obtained by One-way ANOVA with Dunnett's multiple comparisons test or unpaired t-test with Welch's correction. The Kaplan-Meier method and log-rank test were used to compare survival among groups. P<0.05 is considered statistically significant. Data are presented as mean±SEM.

### Example 2

### Combination Therapy With AMD3100 and VIC-008 Augments Tumor Control and Mouse Survival

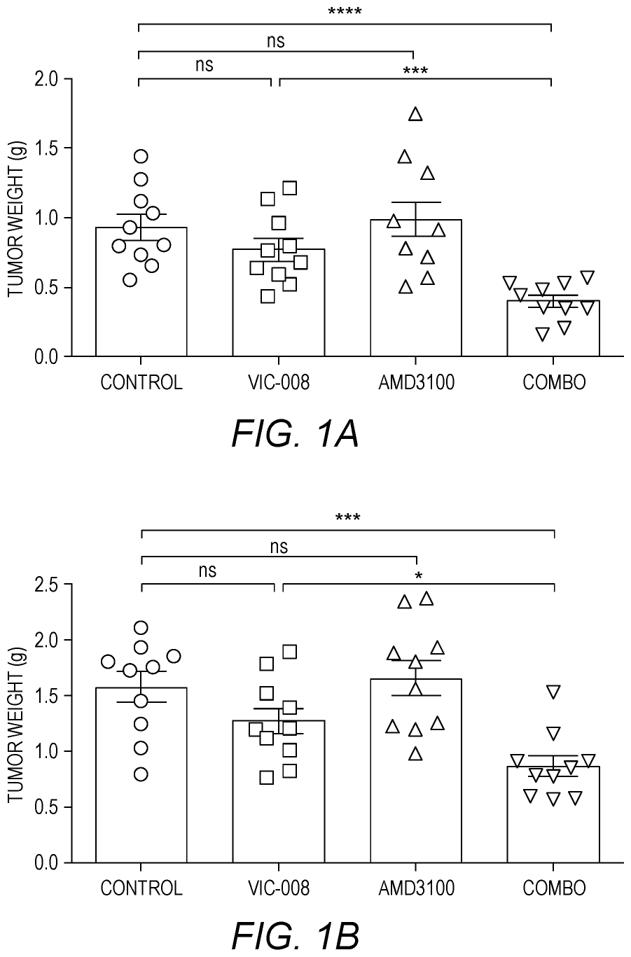

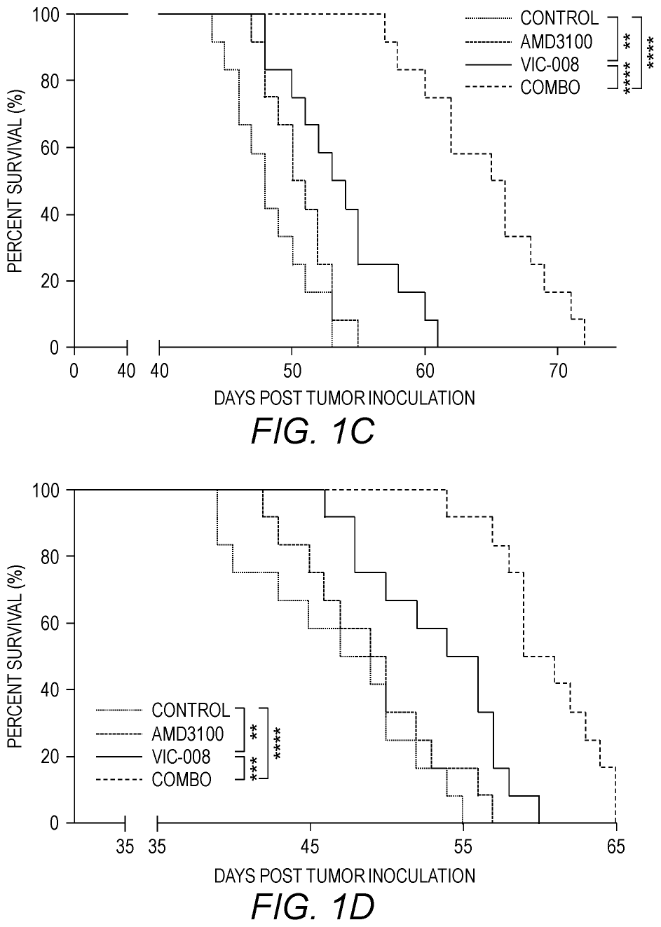

Two intraperitoneal malignant mesothelioma models were established in immunocompetent C57BL/6 mice, separately using the syngeneic 40 L and AE17 cell lines. Here, the effect of AMD3100 and VIC-008 was tested, used singly or in combination, on tumor growth and animal survival in mesothelioma-bearing mice. In animals treated with VIC-008 alone (20 μg per mouse), the total weight of intraperitoneal tumors collected one week after the last treatment was generally reduced (FIGS. 1A-1B) and animal survival was significantly prolonged (FIGS. 1C-1D) compared to saline control treatment in both the 40 L and AE17 models (P<0.01 and P<0.01, respectively). AMD3100 alone at 1 mg/kg of mouse body weight conferred only modest benefit to survival in both 40 L and AE17 mouse MM models compared to saline control treatment. However, the combination treatment with VIC-008 and AMD3100 significantly enhanced tumor control (P<0.0001 and P<0.001, respectively) and prolonged animal survival (P<0.0001 and P<0.0001, respectively) compared to saline control in both 40 L and AE17 models. Moreover, the combination treatment showed further significantly improved antitumor efficacy on inhibition of tumor growth (P<0.001 and P<0.05, respectively) and prolongation of mouse survival (P<0.0001 and P<0.001, respectively) compared to VIC-008 monotherapy in both 40 L and AE17 models. These data indicate that when combined, AMD3100 significantly enhances the antitumor effect of VIC-008 in both 40 L and AE17 MM mouse models compared to monotherapy with either agent.

### Example 3

### VIC-008 Increases Lymphocyte Infiltration in Spleens, Lymph Nodes and Tumors

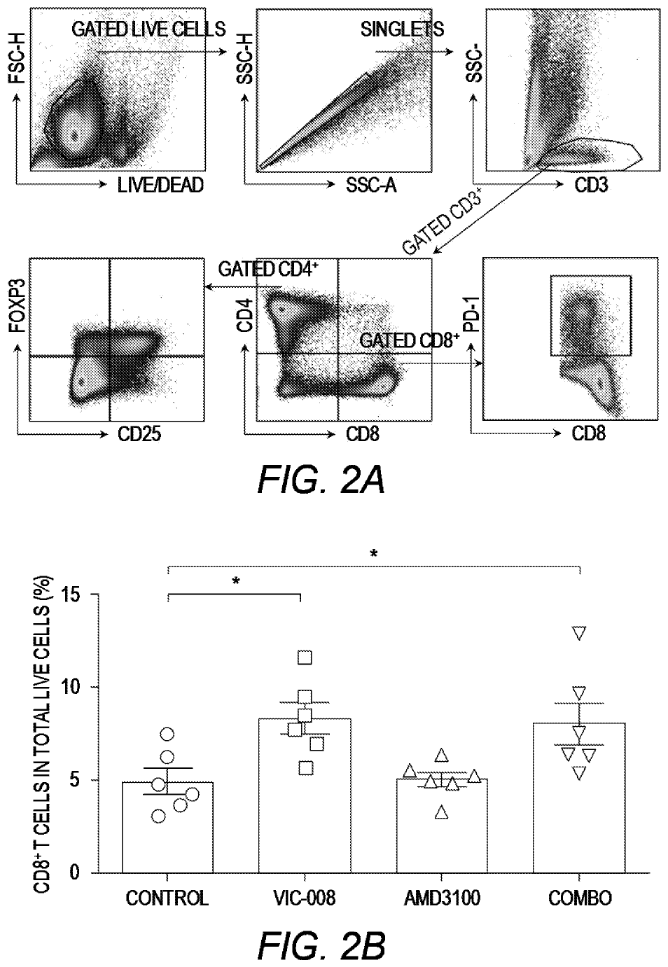

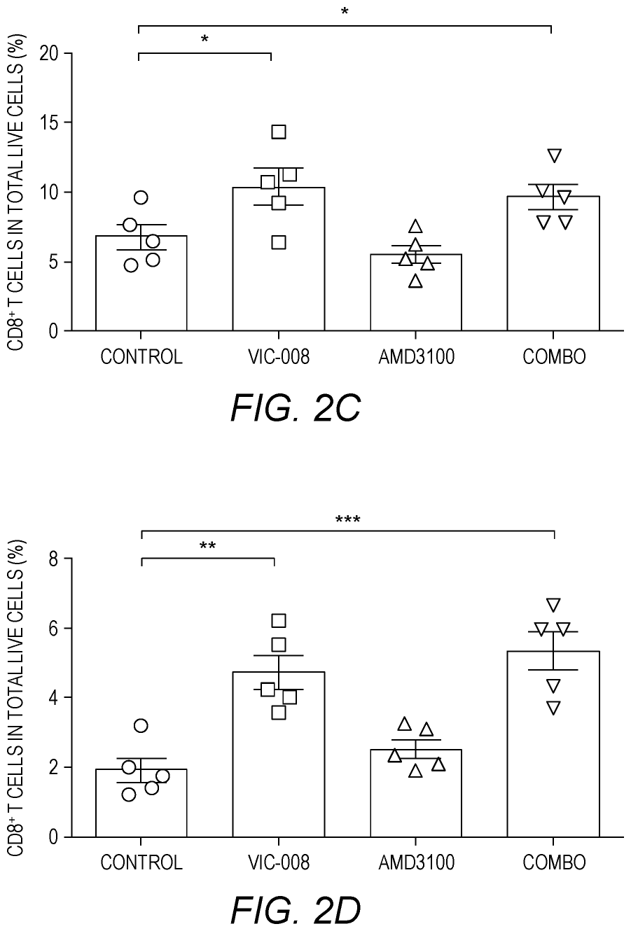

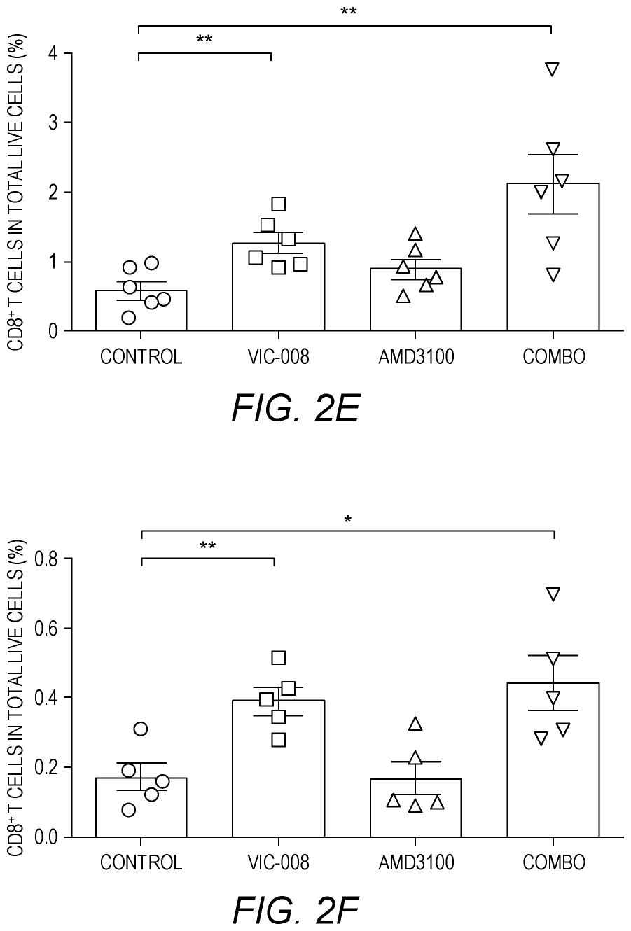

Spleens, axillary and inguinal lymph nodes, and intraperitoneal tumors were collected from tumor-bearing mice one week after the last treatment. Single cells were prepared from these tissues and analyzed by flow cytometry (FIG. 2A). The proportions of CD8⁺ T cells in the total live cells recovered from spleens (P<0.05 and P<0.05, respectively) and tumors (P<0.01 and P<0.01, respectively) for both the 40 L and AE17 models, and from lymph nodes (P<0.01) in AE17 model were significantly increased in the VIC-008 treatment group compared to that in the saline control group (FIGS. 2B-2F). In VIC-008 and AMD3100 combination treatment group, the proportions of CD8⁺ T cells in the total live cells recovered from spleens (P<0.05 and P<0.05, respectively) and tumors (P<0.01 and P<0.05, respectively) for both the 40 L and AE17 models, and from lymph nodes (P<0.001) in AE17 model were significantly increased compared to that in the saline control group. AMD3100 treatment did not increase the proportion of CD8⁺ T cells in these tissues. Moreover, there was no difference in the proportion of CD8⁺ T cells between the VIC-008 and combination treatment groups, indicating that VIC-008 increased lymphocyte infiltration of these tissues.

### Example 4

### VIC-008 Enhances Tumor Antigen-Specific CD8⁺ T-Cell Responses

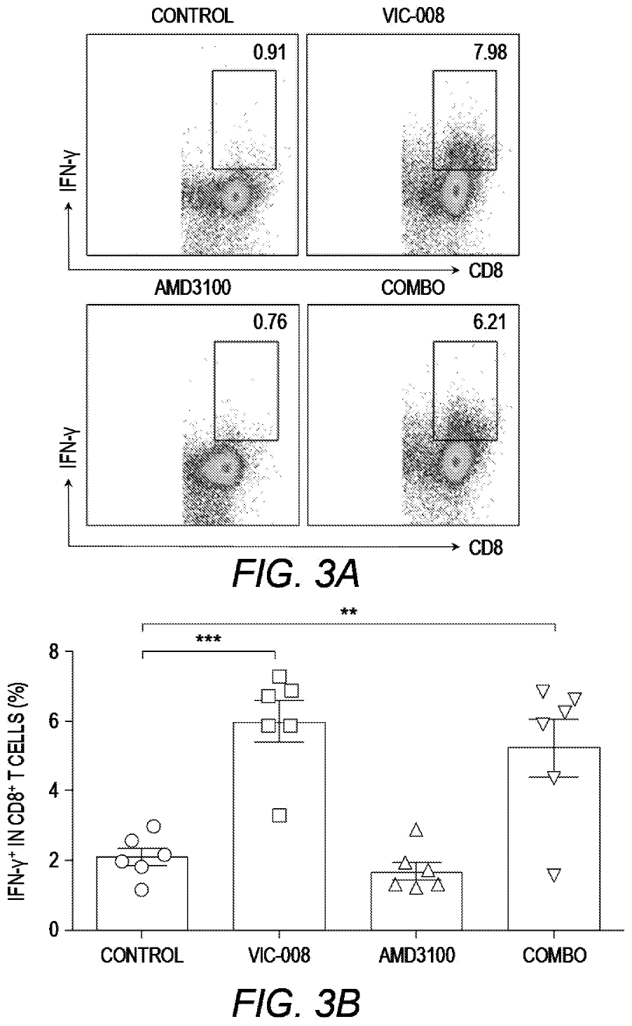

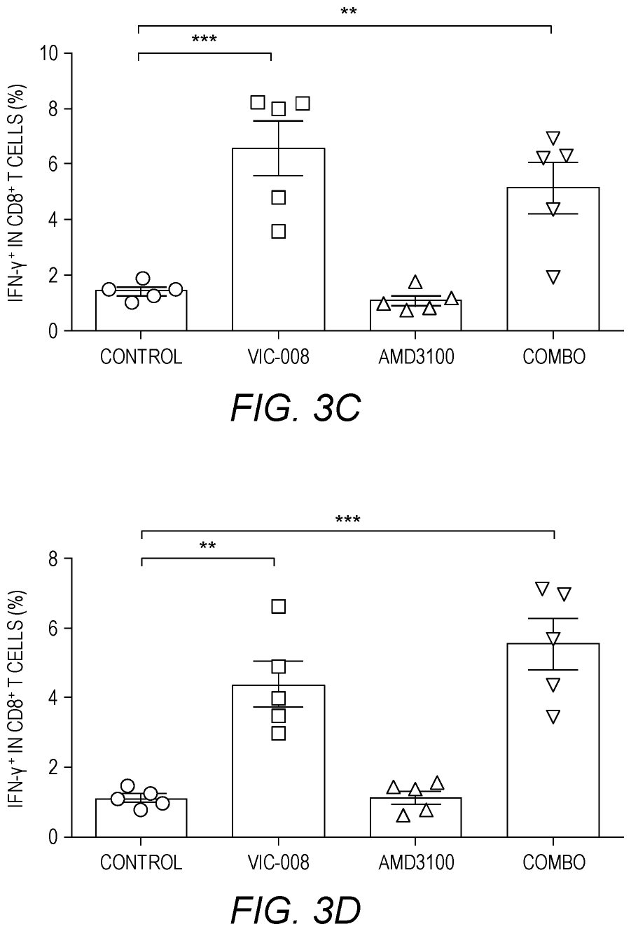

Next, single cells isolated from spleens and lymph nodes from tumor-bearing mice were restimulated with recombinant mesothelin ex vivo and analyzed intracellular IFN-γ in CD8⁺ T cells. Mesothelin-specific IFN-γ expression in splenic CD8⁺ T cells both in 40 L (P<0.001) and AE17 (P<0.001) models, and in lymph node CD8⁺ T cells in the AE17 model (P<0.01), were significantly greater in mice treated with VIC-008 alone compared to that in mice treated with saline (FIGS. 3A-3D). In VIC-008 and AMD3100 combination treatment group, mesothelin-specific IFN-γ expression in splenic CD8⁺ T cells both in 40 L (P<0.01) and AE17 (P<0.01) models, and in lymph node CD8⁺ T cells in the AE17 model (P<0.001), were significantly greater compared to that in mice treated with saline. AMD3100 treatment by itself did not enhance antigen-specific IFN-γ secretion in CD8⁺ T cells. Together, these data support the view that VIC-008 treatment enhances antitumor CD8⁺ T-cell responses in both the 40 L and AE17 mesothelioma mouse models.

### Example 5

### AMD3100 Decreases PD-1 Expression on CD8⁺ T Cells

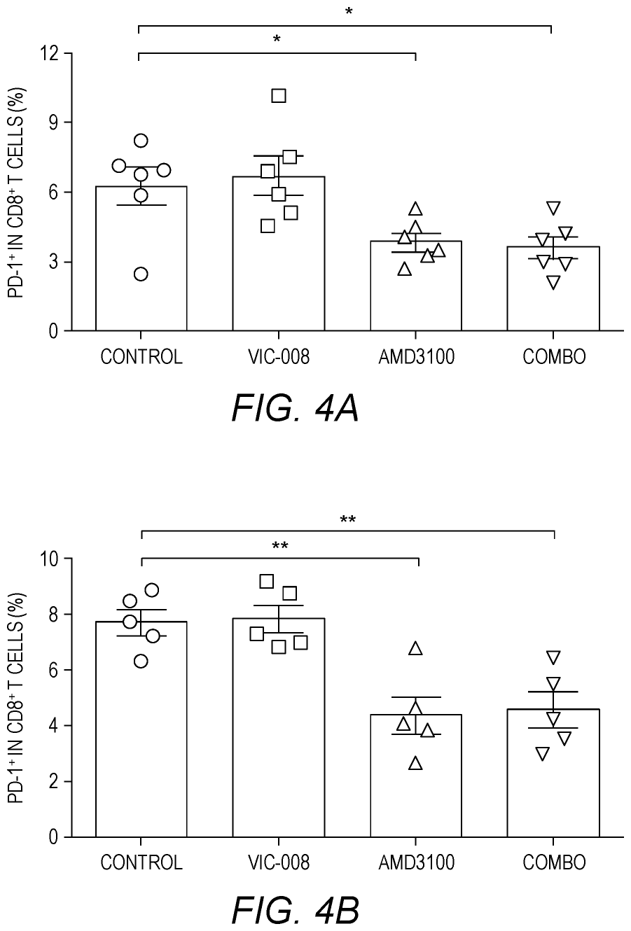

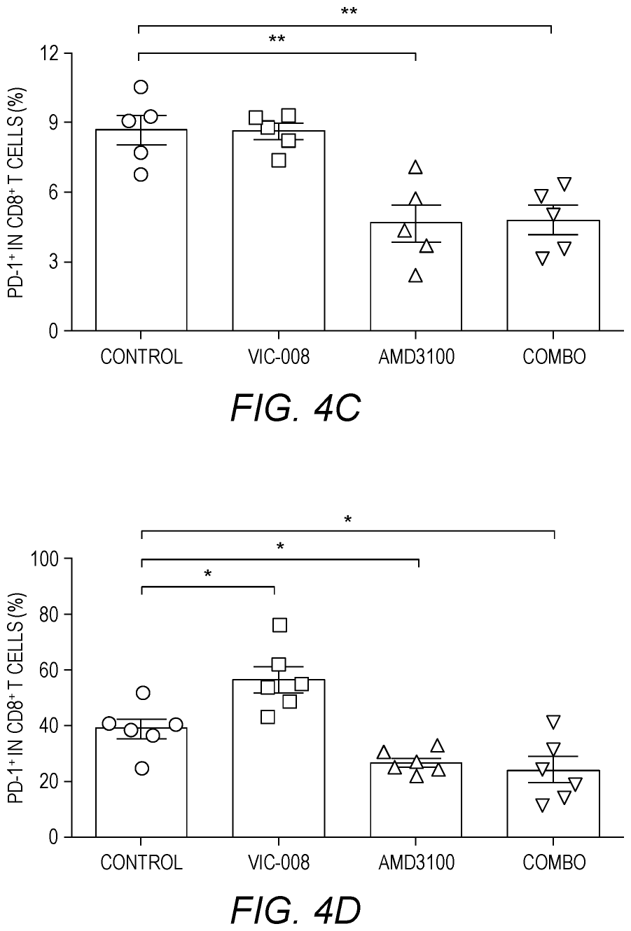

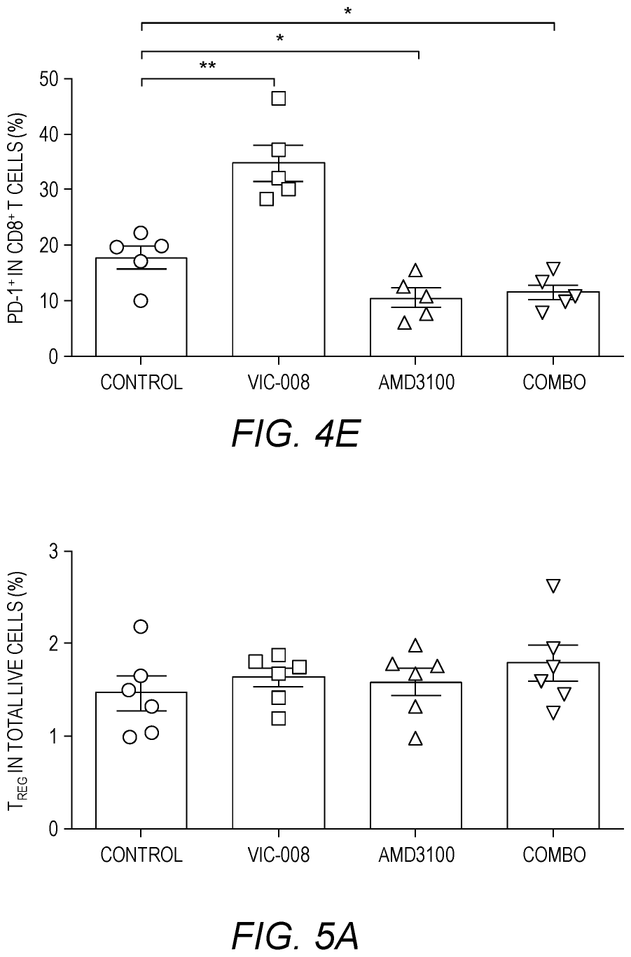

Next, expression of programmed cell death protein-1 (PD-1) on CD8⁺ T cells was evaluated in spleen, tumor and lymph nodes. There was no significant difference between the VIC-008 treatment group and the saline control group in the proportion of PD-1-expressing CD8⁺ T cells in spleens in both the 40 L and AE17 tumor-bearing mice, and in lymph nodes for AE17 mice (FIGS. 4A-4C). In marked contrast, significantly more intratumoral CD8⁺ T cells in the VIC-008 treated group expressed PD-1 in the 40 L tumors (P<0.05) and AE17 tumors (P<0.01) compared with the saline-treated controls (FIGS. 4D-4E). The percentage of PD-1 expressing CD8⁺ T cells ranged between 43-76% and 28-47% in the 40 L tumors and AE17 tumors respectively compared to only 5-10% in spleen and lymph nodes. These data indicate that the antitumor activity of the CD8⁺ T cells in the tumor environment induced by VIC-008 treatment could be obstructed by activation of the PD-1/PD-L1 pathway.

Compared to this effect of VIC-008, and surprisingly, it was found that AMD3100 reduced PD-1 expression on CD8⁺ T cells. AMD3100 treatment alone led to significantly fewer PD-1-expressing CD8⁺ T cells in spleens (P<0.05 and P<0.01, respectively) and tumors (P<0.05 and P<0.05, respectively) in both the 40 L and AE17 tumor-bearing mice, and in lymph nodes (P<0.01) for AE17 mice than in mice treated with saline. In AMD3100 and VIC-008 combination treatment group significantly fewer CD8⁺ T cells expressed PD-1 in spleens (P<0.05 and P<0.01, respectively) and tumors (P<0.05 and P<0.05, respectively) in both the 40 L and AE17 tumor-bearing mice, and in lymph nodes (P<0.01) for AE17 mice compared with the saline-treated controls. There was no significant difference in the proportion of PD-1-expressing CD8⁺ T cells in these tissues between the AMD3100 monotherapy and AMD3100-VIC-008 combination therapy groups. These data indicated that AMD3100 could inhibit CD8⁺ T cells from expressing PD-1 in spleens, lymph nodes and tumors.

### Example 6

### AMD3100 Reduces Tumor-Infiltrating T_{reg} Cells

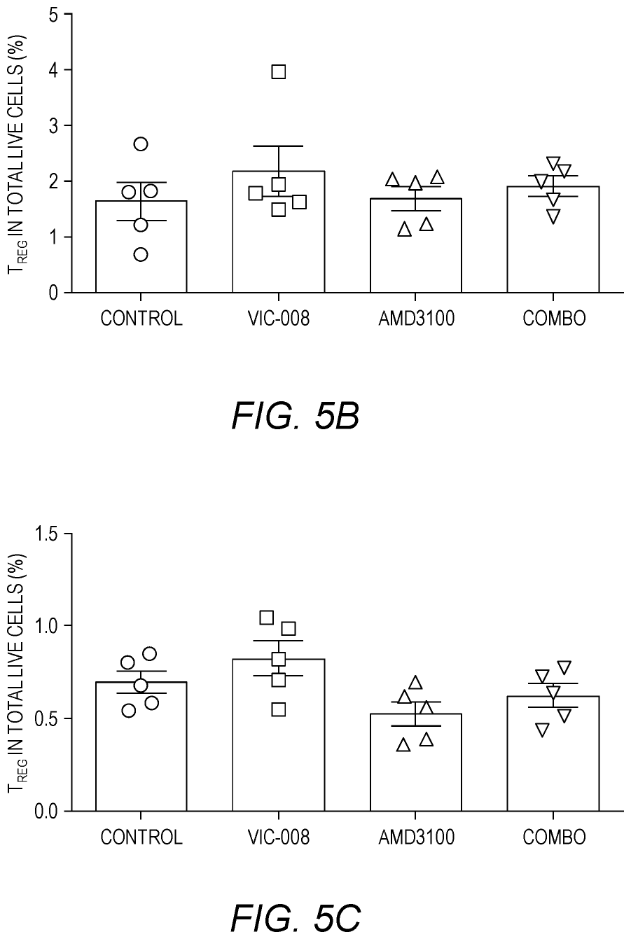

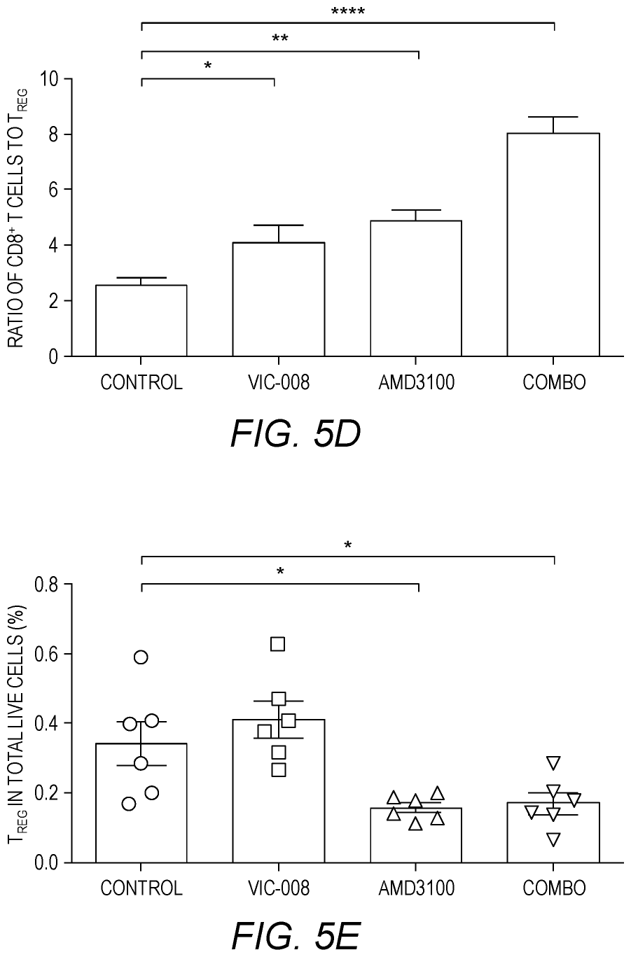

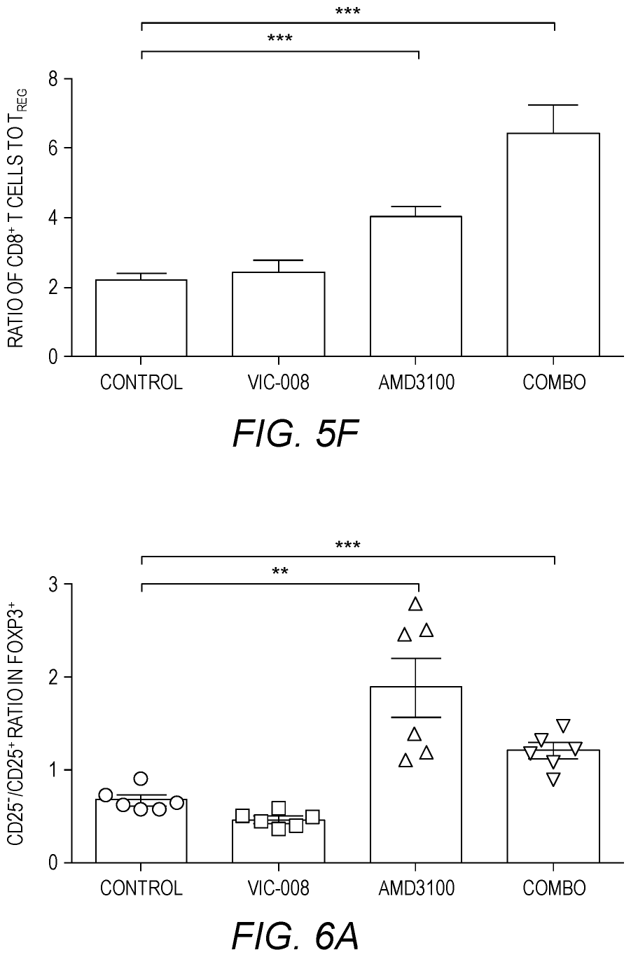

Next, the impact of AMD3100 on T_{reg} cells was evaluated. AMD3100 did not alter the proportions of T_{reg} cells found in spleens of 40 L tumor-bearing mice (FIGS. 5A-5B). In AE17 tumor-bearing mice AMD3100 alone generally reduced T_{reg} in the lymph nodes (FIG. 5C) and AMD3100 alone or in combination with VIC-008 significantly increased the cell ratio of CD8⁺ T cells to T_{reg} cells (P<0.01 and P<0.0001, respectively) compared to saline treatment (FIG. 5D). In tumors from both the 40 L and AE17 models, AMD3100 applied as monotherapy significantly decreased the proportions of T_{reg} (P<0.05 and P<0.01, respectively) and increased the ratio of CD8⁺ T cells to T_{reg} (P<0.001 and P<0.01, respectively) compared to saline treatment (FIGS. 5E-5F). In AMD3100 and VIC-008 combination treatment group the proportions of T_{reg} were significantly decreased (P<0.05 and P<0.05, respectively) and the ratio of CD8⁺ T cells to T_{reg} increased (P<0.001 and P<0.0001, respectively) compared to saline treatment in both the 40 L and AE17 models. In these two murine mesothelioma models, AMD3100 reduced intratumoral T_{reg} infiltration.

### Example 7

### AMD3100 Modulates T_{reg} Cells Toward a T Helper Phenotype

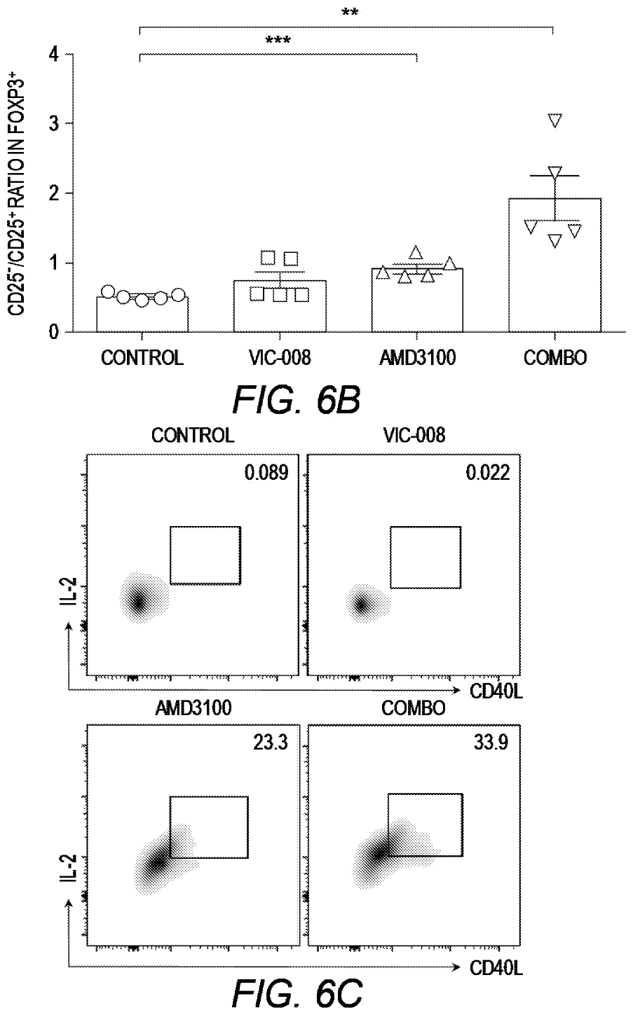

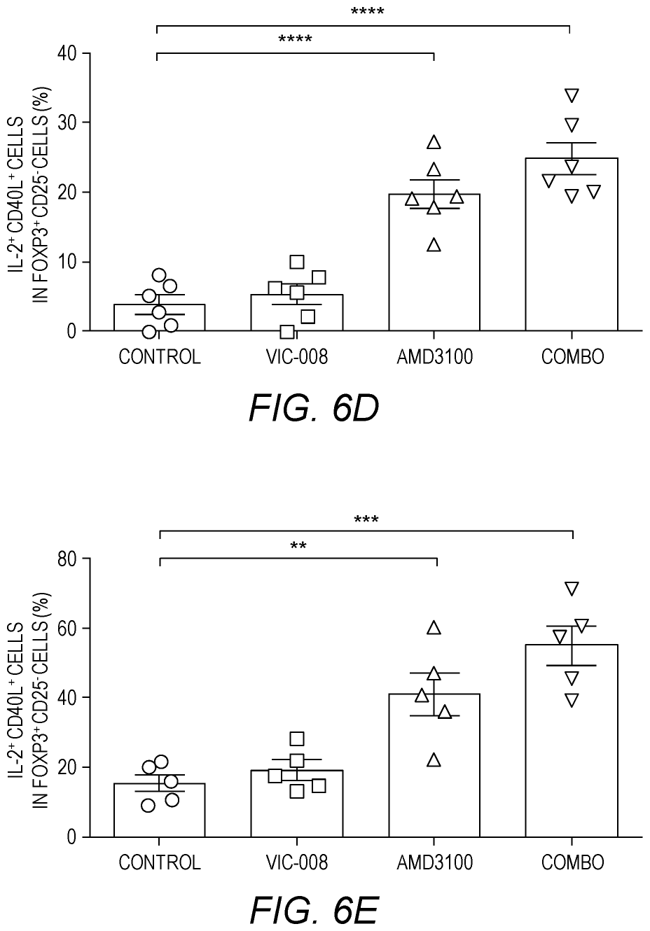

It was observed that AMD3100, alone or in combination with VIC-008, significantly increased the ratio of CD25⁻ cells to CD25⁺ cells within the Foxp3⁺ population in both 40 L (FIG. 6A, P<0.01 and P<0.001, respectively) and in the lymph nodes in the AE17 model (FIG. 6B, P<0.001 and P<0.01, respectively). Among the Foxp3⁺ CD25⁻ T_{reg} population significantly more cells were phenotypically IL-2⁺ CD40L⁺ (FIGS. 6C-6E) after AMD3100 monotherapy and combination therapy with VIC-008, which suggested a change from T_{reg} cells to helper-like cells that had lost CD25 without loss of Foxp3, and may have lost their immunosuppressive function. There was no difference in the proportion of IL-2⁺ CD40L⁺ cells in the Foxp3⁺ CD25⁻ T_{reg} population between AMD3100 monotherapy and combination therapy groups, indicating that AMD3100 treatment may be the major driver of reprogramming of T_{reg} into helper-like cells.

### Example 8

### AMD3100-Driven Modulation of T_{reg} Phenotype Requires TCR Activation

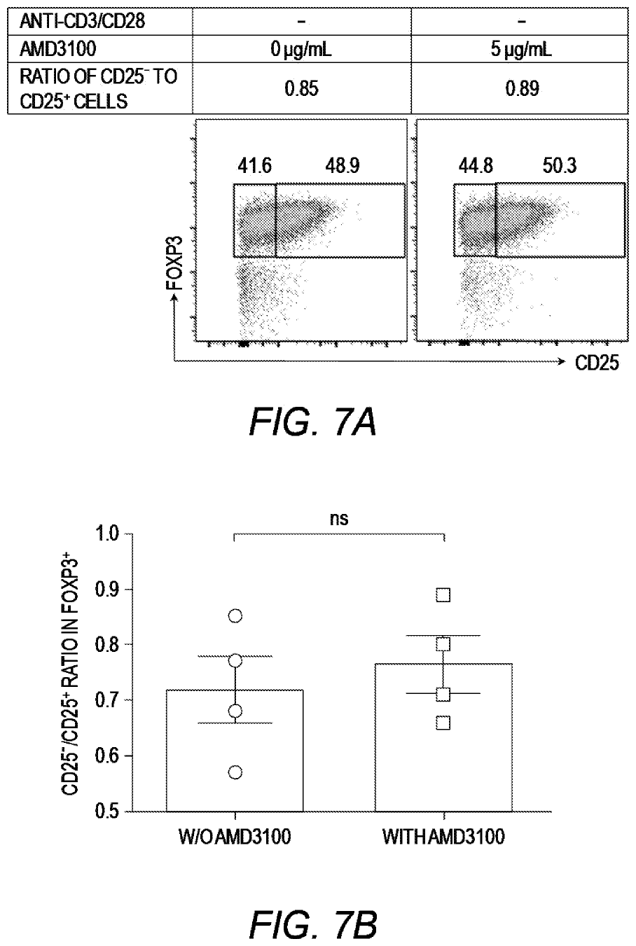

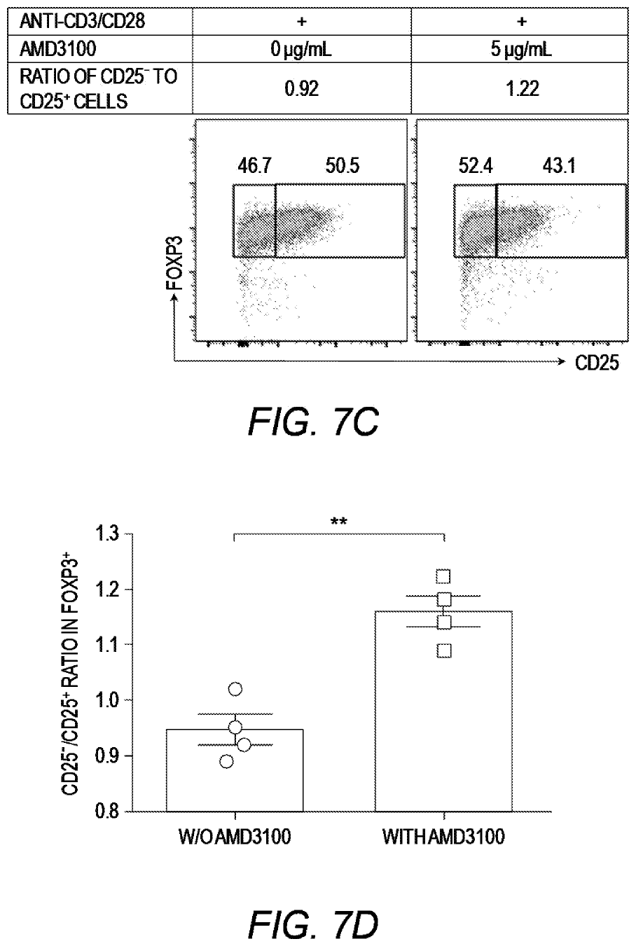

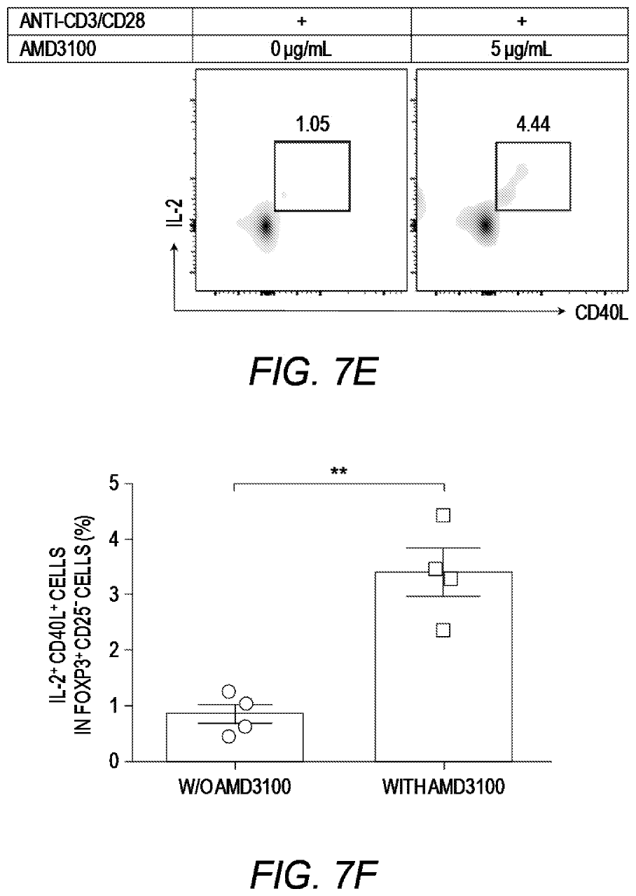

Next, it was addressed whether AMD3100-driven T_{reg} modulation could be initiated in isolated single cells. Cells expressing GFP-Foxp3 from CD4⁺ splenocytes in T-Red/FoxP3 GFP transgenic mice were sorted and treated in vitro with AMD3100. AMD3100 treatment alone did not change the ratio of the proportion of CD25⁻ cells to CD25⁺ cells in the Foxp3⁺ CD4⁺ population (FIGS. 7A-7B). However, in the presence of stimulation by anti-CD3/CD28 antibodies to trigger TCR activation, AMD3100 treatment significantly increased the ratio of the proportion of CD25⁻ cells to CD25⁺ cells in the Foxp3⁺ CD4⁺ population (FIGS. 7C-7D, P=0.0017) and converted more Foxp3⁺ CD25⁻ T_{reg} into IL-2⁺ CD40L⁺ cells (FIGS. 7E-7F, P=0.0015). These data indicated that the conversion of T_{reg} cells into helper-like cells can be mediated by AMD3100 treatment of single cells upon TCR activation.

MSLN is highly overexpressed on the surface of a number of common epithelial cancers including epithelial MM, while expressed only at relatively low levels in normal mesothelial cells lining the pleura, pericardium, and peritoneum in healthy individuals (34-36). MtbHsp70 is well characterized and functions as a potent immune adjuvant (37). It stimulates monocytes and dendritic cells (DCs) to produce CC-chemokines (38, 39), which attract antigen processing and presenting macrophages, DCs, and effector T and B cells (40). Antigenic peptides linked to MtbHsp70 can elicit both MHC class I-restricted CD8⁺ and MHC class II-restricted CD4⁺ T-cell responses (41-45). The fusion of the anti-MSLN scFv and MtbHsp70 takes advantage of the immune-activating action of MtbHsp70 and the tumor-targeting activity of the scFv to promote antitumor responses against the broadest profile of tumor antigens. Consistent with previous findings in a mouse model of ovarian cancer, here it is demonstrated that VIC-008 enhanced tumor-specific CD8⁺ T cell-mediated cytotoxic responses and facilitated lymphocyte intratumoral infiltration in mouse models of MM, resulting in significant prolongation of animal survival in spite of modest control of tumor growth. However, it was also found that PD-1 expression was significantly upregulated on tumor-infiltrated CD8⁺ T cells in VIC-008 treated mice, indicating that antitumor activity of VIC-008-induced CD8⁺ T cells could be compromised by PD-1/PD-L1 pathway activation.

CXCL12 and its cognate receptor CXCR4 constitute a chemokine-receptor axis that is known to be overexpressed in the tumor microenvironment of various cancers, and activation of the CXCL12-CXCR4 axis is associated with disease progression (19, 20). In vivo and in vitro models have revealed that AMD3100, an antagonist of CXCR4, can inhibit tumor growth, metastasis and angiogenesis by blockade of CXCL12-CXCR4 interaction and the subsequent inhibition of PI3K-Akt or Ras/Raf-Erk1/2 signaling (22, 23). The CXCL12-CXCR4 axis is also known to mediate trafficking and retention of various immune cells at specific anatomic sites (46, 47). T_{reg} cells are thought to modulate antitumor immune responses through selective migration to and accumulative retention at tumor sites, thereby playing an important role in the immunopathogenesis of tumors (48). For example, basal-like breast cancers behave more aggressively despite the presence of dense lymphoid infiltration due to T_{reg} recruitment driven by hypoxia-induced up-regulation of CXCR4 in T_{reg} cells (49). It has been reported that in an environment of elevated CXCL12 levels in an ovarian cancer model, blockade of the CXCL12/CXCR4 axis with AMD3100 elicited multimodal effects on tumor pathogenesis, including selective reduction of intratumoral T_{reg} cells and release of antitumor effector T cells from immunosuppression, and conferred a significant survival advantage to the ovarian tumor bearing mice (23). In the present study, it was demonstrated for the first time, to our knowledge, that AMD3100 promoted the conversion of phenotypically suppressive CD25⁺Foxp3⁺ T_{reg} cells to CD25⁻ Foxp3⁺ IL-2⁺ CD40L⁺ helper-like cells (31), which may result in the loss of immunosuppressive function of intratumoral T_{reg} cells (32, 33). CD25⁻ Foxp3⁺ IL-2⁺ CD40L⁺ helper-like cells have been demonstrated to have rapid-acting supportive roles in priming CD8⁺ T-cell responses, but they need initial signals from activated CD8⁺ T-cells to initiate the reprogramming (31, 50). Thus, it is hypothesized that in the combinatorial treatment setting VIC-008-activated CD8⁺ T-cell responses further facilitate AMD3100-mediated T_{reg} reprogramming, contributing to the observed enhanced antitumor efficacy.

Recent studies have demonstrated that inhibition of CXCR4 signaling restores sensitivity to CTLA-4 and PD-1 checkpoint inhibitors (21, 22), suggesting a new mechanism by which AMD3100 may modulate immune responses. Indeed, in this study it was found that AMD3100 was associated with suppression of PD-1 expression on CD8⁺ T cells, consistent with abrogation of PD-1-mediated immunosuppression. Furthermore, this study demonstrated that the augmentation of tumor-specific CD8⁺ T cell responses induced by VIC-008, together with the abrogation of immunosuppression mediated by AMD3100, both through inhibition of PD-1 expression on intratumoral CD8⁺ T cells and through conversion of suppressive T_{reg} to helper-like cells, confers significant benefits on tumor control and animal survival in MM.

These data reveal a new dimension of the therapeutic potential of AMD3100, highlighting its potential to act as a potent immune modulator that can complement the activity of targeted cancer immunotherapies such as DC or T-cell based vaccines. In this way this study may offer a new therapeutic approach for significantly prolonging the survival of patients with MM, and expand the potential clinical efficacy of both novel and established DC or T-cell based vaccines and immunotherapies for this therapeutically challenging disease.

### REFERENCES

1. Skammeritz E, Omland L H, Johansen J P, Omland O. Asbestos exposure and survival in malignant mesothelioma: a description of 122 consecutive cases at an occupational clinic. The international journal of occupational and environmental medicine. 2011; 2(4):224-36.
2. Zucali P A, Ceresoli G L, De Vincenzo F, Simonelli M, Lorenzi E, Gianoncelli L, et al. Advances in the biology of malignant pleural mesothelioma. Cancer treatment reviews. 2011; 37(7):543-58.
3. Brims F J. Asbestos—a legacy and a persistent problem. Journal of the Royal Naval Medical Service. 2009; 95(1):4-11.
4. Sekido Y. Molecular pathogenesis of malignant mesothelioma. Carcinogenesis. 2013; 34(7):1413-9.
5. Mossman B T, Shukla A, Heintz N H, Verschraegen C F, Thomas A, Hassan R. New Insights into Understanding the Mechanisms, Pathogenesis, and Management of Malignant Mesotheliomas. Am J Pathol. 2013; 182(4):1065-77.
6. Cornelissen R, Lievense L A, Heuvers M E, Maat A P, Hendriks R W, Hoogsteden H C, et al. Dendritic cell-based immunotherapy in mesothelioma. Immunotherapy. 2012; 4(10):1011-22.
7. Cornelissen R, Heuvers M E, Maat A P, Hendriks R W, Hoogsteden H C, Aerts J G, et al. New roads open up for implementing immunotherapy in mesothelioma. Clinical & developmental immunology. 2012; 2012:927240.
8. Thapa B, Watkins D N, John T. Immunotherapy for malignant mesothelioma: reality check. Expert Rev Anticancer Ther. 2016:1-10.
9. Yuan J, Kashiwagi S, Reeves P, Nezivar J, Yang Y, Arrifin N H, et al. A novel mycobacterial Hsp70-containing fusion protein targeting mesothelin augments antitumor immunity and prolongs survival in murine models of ovarian cancer and mesothelioma. J Hematol Oncol. 2014; 7:15.
10. Hassan R, Cohen S J, Phillips M, Pastan I, Sharon E, Kelly R J, et al. Phase I clinical trial of the chimeric anti-mesothelin monoclonal antibody MORAb-009 in patients with mesothelin-expressing cancers. Clinical cancer research: an official journal of the American Association for Cancer Research. 2010; 16(24):6132-8.
11. Hassan R, Ho M. Mesothelin targeted cancer immunotherapy. Eur J Cancer. 2008; 44(1):46-53.
12. Kreitman R J, Hassan R, Fitzgerald D J, Pastan I. Phase I trial of continuous infusion anti-mesothelin recombinant immunotoxin SS1P. Clinical cancer research: an official journal of the American Association for Cancer Research. 2009; 15(16):5274-9.
13. Zeng Y L, B.; Reeves, P.; Ran, C.; Liu, Z.; Agha-Abbaslou, M.; Yuan, J.; Leblanc, P.; Sluder, A.; Gelfand, J.; Brauns, T.; Poznansky, P.; Chen, H. Improved antitumoral efficacy of mesothelin targeted immune activating fusion protein in murine model of ovarian cancer. Int J Cancer Clin Res. 2016; 3:051.
14. Ireland D J, Kissick H T, Beilharz M W. The Role of Regulatory T Cells in Mesothelioma. Cancer microenvironment: official journal of the International Cancer Microenvironment Society. 2012.
15. Guo F, Wang Y, Liu J, Mok S C, Xue F, Zhang W. CXCL12/CXCR4: a symbiotic bridge linking cancer cells and their stromal neighbors in oncogenic communication networks. Oncogene. 2016; 35(7):816-26.
16. Domanska U M, Kruizinga R C, Nagengast W B, Timmer-Bosscha H, Huls G, de Vries E G, et al. A review on CXCR4/CXCL12 axis in oncology: no place to hide. Eur J Cancer. 2013; 49(1):219-30.
17. Donzella G A, Schols D, Lin S W, Este J A, Nagashima K A, Maddon P J, et al. AMD3100, a small molecule inhibitor of HIV-1 entry via the CXCR4 co-receptor. Nat Med. 1998; 4(1):72-7.
18. Liles W C, Broxmeyer H E, Rodger E, Wood B, Hubel K, Cooper S, et al. Mobilization of hematopoietic progenitor cells in healthy volunteers by AMD3100, a CXCR4 antagonist. Blood. 2003; 102(8):2728-30.
19. Muller A, Homey B, Soto H, Ge N, Catron D, Buchanan M E, et al. Involvement of chemokine receptors in breast cancer metastasis. Nature. 2001; 410(6824):50-6.
20. Marchesi F, Monti P, Leone B E, Zerbi A, Vecchi A, Piemonti L, et al. Increased survival, proliferation, and migration in metastatic human pancreatic tumor cells expressing functional CXCR4. Cancer research. 2004; 64(22):8420-7.
21. Scala S. Molecular Pathways: Targeting the CXCR4-CXCL12 Axis—Untapped Potential in the Tumor Microenvironment. Clinical cancer research: an official journal of the American Association for Cancer Research. 2015; 21(19):4278-85.
22. Chen Y, Ramjiawan R R, Reiberger T, Ng M R, Hato T, Huang Y, et al. CXCR4 inhibition in tumor microenvironment facilitates anti-programmed death receptor-1 immunotherapy in sorafenib-treated hepatocellular carcinoma in mice. Hepatology. 2015; 61(5):1591-602.
23. Righi E, Kashiwagi S, Yuan J, Santosuosso M, Leblanc P, Ingraham R, et al. CXCL12/CXCR4 blockade induces multimodal antitumor effects that prolong survival in an immunocompetent mouse model of ovarian cancer. Cancer research. 2011; 71(16):5522-34.
24. Berzofsky J A, Terabe M, Wood L V. Strategies to Use Immune Modulators in Therapeutic Vaccines Against Cancer. Seminars in oncology. 2012; 39(3):348-57.
25. Berzofsky J A, Ahlers J D, Janik J, Morris J, Oh S, Terabe M, et al. Progress on new vaccine strategies against chronic viral infections. The Journal of clinical investigation. 2004; 114(4):450-62.
26. Berzofsky J A, Terabe M, Oh S, Belyakov I M, Ahlers J D, Janik J E, et al. Progress on new vaccine strategies for the immunotherapy and prevention of cancer. The Journal of clinical investigation. 2004; 113(11):1515-25.
27. Sakaguchi S. Naturally arising CD4+ regulatory t cells for immunologic self-tolerance and negative control of immune responses. Annual review of immunology. 2004; 22:531-62.
28. Terabe M, Berzofsky J A. Immunoregulatory T cells in tumor immunity. Current opinion in immunology. 2004; 16(2):157-62.
29. Kusmartsev S, Gabrilovich D I. Role of immature myeloid cells in mechanisms of immune evasion in cancer. Cancer immunology, immunotherapy: CII. 2006; 55(3):237-45.
30. Cordier Kellerman L, Valeyrie L, Fernandez N, Opolon P, Sabourin J C, Maubec E, et al. Regression of AK7 malignant mesothelioma established in immunocompetent mice following intratumoral gene transfer of interferon gamma. Cancer Gene Ther. 2003; 10(6):481-90.
31. Sharma M D, Huang L, Choi J H, Lee E J, Wilson J M, Lemos H, et al. An inherently bifunctional subset of Foxp3+ T helper cells is controlled by the transcription factor eos. Immunity. 2013; 38(5):998-1012.
32. Nishikawa H, Sakaguchi S. Regulatory T cells in cancer immunotherapy. Current opinion in immunology. 2014; 27:1-7.
33. Sharma M D, Baban B, Chandler P, Hou D Y, Singh N, Yagita H, et al. Plasmacytoid dendritic cells from mouse tumor-draining lymph nodes directly activate mature Tregs via indoleamine 2,3-dioxygenase. The Journal of clinical investigation. 2007; 117(9):2570-82.
34. Chang K, Pastan I. Molecular cloning of mesothelin, a differentiation antigen present on mesothelium, mesotheliomas, and ovarian cancers. Proceedings of the National Academy of Sciences of the United States of America. 1996; 93(1):136-40.
35. Argani P, Iacobuzio-Donahue C, Ryu B, Rosty C, Goggins M, Wilentz R E, et al. Mesothelin is overexpressed in the vast majority of ductal adenocarcinomas of the pancreas: identification of a new pancreatic cancer marker by serial analysis of gene expression (SAGE). Clinical cancer research: an official journal of the American Association for Cancer Research. 2001; 7(12):3862-8.
36. Ho M, Bera T K, Willingham M C, Onda M, Hassan R, FitzGerald D, et al. Mesothelin expression in human lung cancer. Clinical cancer research: an official journal of the American Association for Cancer Research. 2007; 13(5):1571-5.
37. Bulut Y, Michelsen K S, Hayrapetian L, Naiki Y, Spallek R, Singh M, et al. Mycobacterium tuberculosis heat shock proteins use diverse Toll-like receptor pathways to activate pro-inflammatory signals. J Biol Chem. 2005; 280(22):20961-7.
38. Floto R A, MacAry P A, Boname J M, Mien T S, Kampmann B, Hair J R, et al. Dendritic cell stimulation by mycobacterial Hsp70 is mediated through CCRS. Science. 2006; 314(5798):454-8.
39. Wang Y, Kelly C G, Karttunen J T, Whittall T, Lehner P J, Duncan L, et al. CD40 is a cellular receptor mediating mycobacterial heat shock protein 70 stimulation of CC-chemokines. Immunity. 2001; 15(6):971-83.
40. Baggiolini M. Chemokines and leukocyte traffic. Nature. 1998; 392(6676):565-8.
41. Udono H, Srivastava P K. Heat shock protein 70-associated peptides elicit specific cancer immunity. The Journal of experimental medicine. 1993; 178(4):1391-6.
42. Suto R, Srivastava P K. A mechanism for the specific immunogenicity of heat shock protein-chaperoned peptides. Science. 1995; 269(5230):1585-8.
43. Suzue K, Young R A. Adjuvant-free hsp70 fusion protein system elicits humoral and cellular immune responses to HIV-1 p24. J Immunol. 1996; 156(2):873-9.
44. Huang Q, Richmond J F, Suzue K, Eisen H N, Young R A. In vivo cytotoxic T lymphocyte elicitation by mycobacterial heat shock protein 70 fusion proteins maps to a discrete domain and is CD4(+) T cell independent. The Journal of experimental medicine. 2000; 191(2):403-8.
45. Ciupitu A-M T, Petersson M, O'Donnell C L, Williams K, Jindal S, Kiessling R, et al. Immunization with a Lymphocytic Choriomeningitis Virus Peptide Mixed with Heat Shock Protein 70 Results in Protective Antiviral Immunity and Specific Cytotoxic T Lymphocytes. The Journal of experimental medicine. 1998; 187(5):685-91.
46. Luster A D. The role of chemokines in linking innate and adaptive immunity. Current opinion in immunology. 2002; 14(1):129-35.
47. Vianello F, Papeta N, Chen T, Kraft P, White N, Hart W K, et al. Murine B16 melanomas expressing high levels of the chemokine stromal-derived factor-1/CXCL12 induce tumor-specific T cell chemorepulsion and escape from immune control. J Immunol. 2006; 176(5):2902-14.
48. Zou W Immunosuppressive networks in the tumour environment and their therapeutic relevance. Nature reviews Cancer. 2005; 5(4):263-74.
49. Yan M, Jene N, Byrne D, Millar E K, O'Toole S A, McNeil C M, et al. Recruitment of regulatory T cells is correlated with hypoxia-induced CXCR4 expression, and is associated with poor prognosis in basal-like breast cancers. Breast cancer research: BCR. 2011; 13(2):R47.
50. Sharma M D, Shinde R, McGaha T L, Huang L, Holmgaard R B, Wolchok J D, et al. The PTEN pathway in Tregs is a critical driver of the suppressive tumor microenvironment. Sci Adv. 2015; 1(10):e1500845.

The foregoing is illustrative of the present invention, and is not to be construed as limiting thereof. The invention is defined by the following claims, with equivalents of the claims to be included therein.

That which is claimed is:

1. A method of decreasing the level of PD-1 on a CD8⁺ T cell, comprising contacting the T cell with an amount of an inhibitor of CXCL12 signaling effective to decrease the level of PD-1 on the CD8⁺ T cell.

2. The method of claim 2, wherein the inhibitor of CXCL12 signaling downregulates the expression of PD-1.

3. The method of claim 1 or 2, wherein the CD8⁺ T cell is an in vitro or ex vivo cell.

4. The method of claim 3, wherein contacting the T cell comprises co-incubating the T cell and the inhibitor of CXCL12 signaling.

5. The method of claim 1 or 2, wherein the CD8⁺ T cell is in a subject.

6. The method of claim 5, wherein contacting the T cell comprises administering the inhibitor of CXCL12 signaling to the subject.

7. The method of claim 6, wherein the inhibitor of CXCL12 signaling is administered systemically.

8. The method of claim 6, wherein the inhibitor of CXCL12 signaling is administered locally.

9. The method of any one of claims 5-8, wherein the subject is in need of or undergoing immunotherapy.

10. The method of any one of claims 5-8, wherein the subject has failed immunotherapy.

11. The method of any one of claims 5-10, wherein the subject has cancer.

12. The method of any one of claims 5-10, wherein the subject has an infectious disease.

13. The method of any one of claims 5-12, further comprising administering to the subject an immunotherapy or vaccine treatment.

14. The method of any one of claims 1-13, wherein the inhibitor of CXCL12 signaling is an agent that inhibits binding of CXCL12 to CXCR4 or CXCR7.

15. The method of any one of claims 1-14, wherein the inhibitor of CXCL12 signaling is a CXCR4 antagonist and/or a CXCR7 antagonist.

16. The method of claim 15, wherein the CXCR4 antagonist is AMD3100.

17. The method of claim 15, wherein the CXCR4 and/or CXCR7 antagonist is an antibody that specifically binds CXCR4 or CXCR7.

18. A method of converting a CD25⁺ Foxp3⁺ regulatory T cell to a CD25⁻ Foxp3⁺ helper-like T cell, comprising contacting the T cell with an amount of an inhibitor of CXCL12 signaling effective to convert the CD25⁺ Foxp3⁺ regulatory T cell to a CD25⁻ Foxp3⁺ helper-like T cell.

19. The method of claim 18, wherein the helper-like T cell is CD25⁻ Foxp3⁺ IL-2⁺ CD40L⁺.

20. The method of claim 18 or 19, further comprising activating a T cell receptor on the regulatory T cell.

21. The method of claim 20, wherein activating a T cell receptor comprises contacting the regulatory T cell with an anti-CD3/CD28 antibody.

22. The method of any one of claims 18-21, wherein the regulatory T cell is an in vitro or ex vivo cell.

23. The method of claim 22, wherein contacting the T cell comprises co-incubating the T cell and the inhibitor of CXCL1 signaling.

24. The method of any one of claims 18-21, wherein the regulatory T cell is in a subject.

25. The method of claim 24, wherein contacting the T cell comprises administering the inhibitor of CXCL1 signaling to the subject.

26. The method of claim 25, wherein the inhibitor of CXCL1 signaling is administered systemically.

27. The method of claim 25, wherein the inhibitor of CXCL1 signaling is administered locally.

28. The method of any one of claims 24-27, wherein the subject is in need of or undergoing immunotherapy.

29. The method of any one of claims 24-27, wherein the subject has failed immunotherapy.

30. The method of any one of claims 24-29, wherein the subject has cancer.

31. The method of any one of claims 24-29, wherein the subject has an infectious disease.

32. The method of any one of claims 24-31, further comprising administering to the subject an immunotherapy or vaccine treatment.

33. The method of any one of claims 18-32, wherein the inhibitor of CXCL12 signaling is an agent that inhibits binding of CXCL12 to CXCR4 or CXCR7.

34. The method of any one of claims 18-33, wherein the inhibitor of CXCL12 signaling is a CXCR4 antagonist and/or a CXCR7 antagonist.

35. The method of claim 34, wherein the CXCR4 and/or CXCR7 antagonist is AMD3100.

36. The method of claim 34, wherein the CXCR4 and/or CXCR7 antagonist is an antibody that specifically binds CXCR4 or CXCR7.

37. A method of reprogramming subpopulations of T cells to a phenotype suitable to enhance an immunotherapy treatment in a subject, comprising contacting T cell subpopulations with an amount of an inhibitor of CXCL12 signaling effective to reprogram the subpopulations of T cells to a phenotype suitable to enhance an immunotherapy treatment.

38. The method of claim 37, wherein the T cell subpopulations are in vitro or ex vivo.

39. The method of claim 38, wherein the T cell subpopulations are obtained from the subject, reprogrammed, and administered to the subject.

40. The method of claim 38 or 39, wherein contacting the T cell subpopulations comprises co-incubating the T cell subpopulations and the inhibitor of CXCL12 signaling.

41. The method of claim 37, wherein the T cell subpopulations are in a subject.

42. The method of claim 41, wherein contacting the T cell subpopulations comprises administering the inhibitor of CXCL12 signaling to the subject.

43. The method of claim 42, wherein the inhibitor of CXCL12 signaling is administered systemically.

44. The method of claim 42, wherein the inhibitor of CXCL12 signaling is administered locally.

45. The method of any one of claims 41-44, wherein the subject is in need of or undergoing immunotherapy.

46. The method of any one of claims 41-44, wherein the subject has failed immunotherapy.

47. The method of any one of claims 41-46, wherein the subject has cancer.

48. The method of any one of claims 41-46, wherein the subject has an infectious disease.

49. The method of any one of claims 41-48, further comprising administering to the subject an immunotherapy or vaccine treatment.

50. The method of any one of claims 37-49, wherein the inhibitor of CXCL12 signaling is an agent that inhibits binding of CXCL12 to CXCR4 or CXCR7.

51. The method of any one of claims 37-49, wherein the inhibitor of CXCL12 signaling is a CXCR4 antagonist and/or a CXCR7 antagonist.

52. The method of claim 51, wherein the CXCR4 and/or CXCR7 antagonist is AMD3100.

53. The method of claim 51, wherein the CXCR4 and/or CXCR7 antagonist is an antibody that specifically binds CXCR4 or CXCR7.
